(19) United States
(12) Patent Application Publication
(10) Pub. No.: US 2022/0296736 A1
(43) Pub. Date: Sep. 22, 2022

(54) ADMINISTRATION OF THERAPEUTIC AGENTS TO BRAIN AND OTHER CELLS AND TISSUE

(71) Applicant: The Johns Hopkins University, Baltimore, MD (US)

(72) Inventors: Miroslaw Janowski, Towson, MD (US); Wojciech Lesniak, Owemgs Mills, MD (US); Poitr Walczak, Fulton, MD (US); Martin G. Pomper, Baltimore, MD (US); Chengyan Chu, Baltimore, MD (US); Anna Jablonska, Baltimore, MD (US)

(73) Assignees: The Johns Hopkins University, Baltimore, MD (US); The Johns Hopkins University, Baltimore, MD (US)

(21) Appl. No.: 17/641,417

(22) PCT Filed: Sep. 8, 2020
(86) PCT No.: PCT/US20/49717

§ 371 Date: Mar. 8, 2022

(30) Foreign Application Priority Data
Sep. 8, 2019 (US) 62/897371
Sep. 9, 2019 (US) 62/897502

(51) Int. Cl. A61K 51/10

(52) U.S. Cl. CPC A61K 51/1093

(57) ABSTRACT

Methods are provided for administering and/or assessing a therapeutic agent intraarterially across disrupted blood-brain barrier, systemically or directly to the brain parenchyma in a subject. In a particular aspect, drug infusion parameters can be adjusted based on feedback from real-time MRI and quantitative assessment of brain uptake of the infused therapeutic molecules based on PET imaging.

### PRIORITY CLAIM

This application claims benefits of priority to U.S. Provisional Application No. 62/897,371 filed Sep. 8, 2019, and U.S. Provisional Application No. 62/897,502 filed Sep. 9, 2019, the entire contents of which are incorporated herein by reference.

### GOVERNMENT SUPPORT

This invention was made with government support under grant no. NIH R01NS091100, NIH R01NS09110, R21NS106436 and EB024496.

### FIELD

In one aspect, methods and systems are provided to assess therapeutic agents administered to brain tissue of a subject. In a particular aspect, drug infusion parameters can be adjusted based on feedback from real-time imaging and quantitative assessment of brain uptake of the infused therapeutic molecules based on the imaging.

### BACKGROUND

The blood-brain barrier (BBB) is a highly selective permeability barrier that separates the circulating blood in the brain from the central nervous system and which functions to shield the brain from harmful elements in the blood and cerebrospinal fluid (CSF), while facilitating the exchange of essential amino acids, ions, metabolites, neurotransmitters, oxygen, carbon dioxide, growth factors, and other necessary nutrients and cellular wastes within the brain tissue. Although the BBB has evolved to effectively regulate brain homeostasis and to protect the brain from the harmful effects of unwanted elements in the blood and CSF, such as toxins and bacteria, the BBB also presents a significant challenge in the context of delivering therapeutic agents to the brain.

Neurological disorders and cerebral malignancies continue to be a significant burden to the society, in part due to the blood-brain barrier (BBB), which limits access for most macromolecules circulating in the blood, precluding them from reaching therapeutic concentrations in the central nervous system (CNS). Importantly, another relevant function of BBB is active efflux of molecules from the CNS. Therefore, parenchymal accumulation of neurotherapeutic agents is contingent upon both penetration to the CNS and circumvention of clearance by the BBB.

Therapeutic molecules and antibodies that might otherwise be effective in diagnosis and therapy do not generally cross the BBB in adequate amounts to be effective in treatment. Overcoming the difficulty of delivering such therapeutics—ranging from small molecules, protein therapeutics and antibodies, and nucleic acids—presents a major challenge in the treatment of most brain disorders, including brain cancer and tumors, stroke, Alzheimer's disease, and dementia.

Certain approaches have been developed that significantly improve the efficacy of drug delivery to the brain. See U.S. 2017/0029581.

It would be desirable to have additional improved methods for drug administration to a patient's brain for treating a wide array of disorders, including cancer and neurodegenerative disorders.

### SUMMARY

In one aspect, methods and systems are provided to assess the effects of one or more therapeutic agents administered to brain tissue or central nervous system of a subject.

In another aspect, methods and systems are provided to assess the effects of one or more therapeutic agents administered through the blood-brain barrier (BBB) of a subject.

In particular aspects, method and systems are provided that include imaging of a subject to assess in real time the effects of one or more therapeutic agents administered to brain or central nervous system cells or tissue or to cells or tissue (such as cancer cells) located proximate to brain or central nervous system cells or tissue, including for example administration of a therapeutic agents such as through the blood-brain barrier (BBB) of the subject. Imaging may include for example positron emission tomography (PET) imaging, magnetic resonance imaging (MRI), or optical imaging.

Brain uptake and/or clearance of an administered therapeutic agent may be suitably assessed through the present methods and systems. Such uptake and clearance can be suitably assessed through imaging, including positron-emission tomography (PET) and positron-emission tomography with computed tomography (PET/CT), positron-emission tomography with MRI (PET/MRI), or optical imaging methods including fluorescent and/or multiphoton microscopy. In particular, optical imaging can be employed that intravital imaging such as two-photon microscopy (2M) and three-photon microscopy (3PM).

In particular aspects, methods are provided to assess penetration of a therapeutic agent through a subject's blood-brain barrier. In another aspect, methods are provided to measure or assess the level of clearance from a subject's central nervous system a therapeutic agent that has been administered to a subject brain tissue, including through the subject's blood-brain barrier.

We have shown that quantitative assessment of brain uptake of infused therapeutic molecules can be performed in dynamic fashion based on PET imaging and infusion parameters can be adjusted based on feedback from real-time PET to achieve desirable dose and distribution. See the examples which follow. Optical imaging of administered therapeutic molecules also can be performed with infusion parameters adjusted based on feedback from real-time optical imaging data to achieve desirable dose and distribution

Still further, methods of the invention include adjusting administration parameters of one or more therapeutic agents to a subject based on the assessed effects of administration such as uptake and clearance. Thus, for instance, dosage, rate and/or frequency of administration of one or more therapeutic agents may be adjusted or modified over the course of treatment of a subject.

In a preferred embodiment, the present methods and systems may be used to administer and/or assess a therapeutic agent or a diagnostic agent or a combination thereof to the brain or central nervous system of a subject. The therapeutic agent may be for example any agent suitable for administration to the brain or central nervous system including chemotherapeutic agent or a neurotherapeutic agent. Chemotherapeutic agents include any agents known to be therapeutic against cancers including brain cancers and cancers that have metastasized to the brain. Neurotherapeutic agents include, for example, PDGF, VEGF, dopamine and any agent known to be therapeutic to neurological diseases such as Alzheimer's disease, Parkinson disease, stroke, and the like.

In preferred aspects, methods are provided for treating a subject such as a human, which comprise: (a) administering to a subject one or more therapeutic agents intended to pass through the subject's blood-brain barrier and (b) acquiring magnetic resonance images of the subject's blood-brain barrier to thereby assess delivery, residence and/or efficacy of the administered one or more therapeutic agents.

In particular aspects, the one or more therapeutic agents may be administered to a subject intra-arterially. In other aspects, the one or more therapeutic agents may be administered systemically (intravenous, intraperitoneal, per os).

Various imaging methods and systems may be utilized in the present methods, including for example, x-ray, magnetic-resonance imaging (MRI), chemical exchange saturation transfer MRI, positron-emission tomography (PET), positron-emission tomography with computed tomography (PET/CT), PET/MRI (i.e. with machine that can generate both and combined positron emission tomography (PET), magnetic resonance imaging (MRI) scans) and/or optical imaging. As discussed, optical imaging methods including fluorescent and/or multiphoton microscopy, and in particular, intravital imaging such as two-photon microscopy (2M) and three-photon microscopy (3PM).

In one preferred aspect, placement of a catheter in a subject to deliver agents to and across a subject's blood-brain barrier may be navigated using x-ray; opening (includes disruption) of the blood-brain barrier such as by administration of an opening agent may be assessed by magnetic resonance-imaging or optical imaging such as intravital imaging including two-photon microscopy (2M) and three-photon microscopy (3PM); and pharmacokinetics of administered therapeutic agent(s) may be assessed by positron-emission tomography (PET) or optical imaging such as intravital imaging including two-photon microscopy (2M). These preferred imaging protocols suitably may be conducted with distinct apparatus, or one or more combined apparatus such as a PET/MRI scanner.

In additional aspects, method are provided for administering a therapeutic agent including directly to the brain parenchyma through a needle injection in a subject in need thereof (e.g. a subject suffering from a brain disorder), comprising: (a) administering a therapeutically effective amount of one or more therapeutic agents; and (b) assessing the effects of one or more therapeutic agents.

If desired, the subject blood-brain barrier may be disrupted prior or at the same time as administering the one or more therapeutic agents. The effects of the one or more therapeutic agents are preferably assessed by real-time imaging, including PET imaging, or optical imaging such as intravital imaging including two-photon microscopy (2M) and three-photon fluorescence microscopy.

As discussed, while assessing one or more therapeutic agents that have been administered to a subject, current status of the administered agent(s) such as uptake and clearance may be determined, including in substantially real-time. Administration parameters also may be adjusted such as dosage, rate of administration and the like. For example, dosage and/or rate of administration (such as systemic, intraarterial or intraparenchymal infusion of therapeutic agent) may be increased or decreased by 1, 2, 3, 4, 5, 8, 10, 20, 30, 40 50 percent or more based on PET or other imaging of the subject.

In methods and systems of the invention, the administered therapeutic agents may be imaged-assessed for parameters such as uptake and/or clearance at any of a variety of times with respect to administration. For example, the therapeutic agents may be assessed at the time of administration, or for following administration, for example, at 1, 2, 3, 4, 5, 10, 15, 20, 25, 30, 40, 50, or 60 minutes or more following administration, including from 0.5., 1, 2, 3, 6, 12, 24, 48, 72 or 96 hours or more following administration to assess various aspects of the administered therapeutic agent(s) including extent of clearance of the therapeutic agents from the subject or from the target treatment site.

As discussed, in particular aspects, the subject is suffering from a brain disorder, including a proliferative disorder or a neurological disorder, such as brain damage, brain dysfunction, cranial nerve disorder, autonomic nervous system disorder, seizure disorder, movement disorder, sleep disorder, migraine, a central neuropathy, or a neuropsychiatric illness. In one particular embodiment, the disorder is Alzheimer's disease.

In certain embodiments, the therapeutic agent can be an agent for treating a proliferative disorder. The agent can be a small molecule pharmaceutical, or macromolecule including a wide array of biotechnological drugs such as, a therapeutic antibody and other proteins, a therapeutic nucleic acid molecule, a therapeutic lipid-based molecule, any other molecule or a composition comprising any of same.

In disrupting the blood-brain barrier for administration of a therapeutic agents, a blood-brain barrier opening agent may be employed, for example, one or more hyperosmolar agents, such as mannitol, glycerin, isosorbide, or urea. Other opening agents also can be employed such as one or more such as agents “paralyzing” endothelial cells such as various toxins and venoms such as a scorpion venom (e.g. chlorotoxin), or various other agents, for example peptides and peptidomimetics such as MiniCTX3.

In some embodiments, the blood-brain barrier region that is disrupted for administration of a therapeutic agent may be associated with the basilar artery (i.e., associated with the endothelial cell-coated capillaries that are connected to this arterial region). The region of the blood-brain barrier targeted for local disruption can also include other cranial arteries, including the vertebral artery, the occipital artery, the basilar artery, the superficial temporal artery, the middle cerebral artery, the anterior cerebral artery, the posterior cerebral artery, the ophthalmic artery, and the internal carotid artery as well as arteries branching off the listed above arteries.

The present methods and system may be utilized to administer therapeutic agents to areas of a subject's brain, brain tissue, meningeal tissue, central nervous system tissue and cells, among others, as well as malignancies or unwanted growths (e.g. cancer including solid cancer tumors) associated or proximate to such areas, tissue, cells and organs. Examples of central nervous system cells include, for example but not limited to neuron, neuronal cell, brain cells, glial, astrocyte or neuronal supporting cells.

In certain embodiments, the invention also relates to any and all necessary catheter-related control equipment, pumps, drive systems, electrical and fluid control systems, as well as other separate or integrated systems for measuring and visualizing the method of the invention, e.g., fluoroscopic or other visualization systems, vital sign monitoring systems, and the like.

Other aspects of the invention are disclosed infra.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 (includes FIGS. 1A-1E) shows radiolabeling of BV with ⁸⁹Zr. A and B—Reaction schemes demonstrating conjugation of BV with DFO and its subsequent radiolabeling with 89Zr, C—MALDI-TOF spectra of BV and BVDFO, showing increase of the molecular weight that indicates conjugation on average 3 molecules of DFO with each antibody, D—evaluation of BV and BVDFO biding to VGEF, showing that conjugation of DFO with antibody did not affect its targeting properties, E—SEC chromatograms illustrating co-elution of BV (black line, obtained based on absorbance at 280 nm) and ⁸⁹ZrBVDFO (red line, derived using flow-through radiation detector), indicating successful radiolabeling of BVDFO with ⁸⁹Zr.

FIG. 2 (includes FIGS. 2A-2E) shows dynamics of ⁸⁹ZrBVDFO delivery to the brain with or without BBBO. Representative axial, sagittal and coronal PET-CT images obtained by summing 60 frames acquired during 30 min dynamic scans and fusion with CT acquired immediately after dynamic scans, illustrating brain uptake of ⁸⁹ZrBVDFO upon: A—IA infusion of ˜8.5 MBq (˜230 μCi) of ⁸⁹ZrBVDFO reconstituted in 1 mL of saline at 0.15 mL/min with BBBI, B—BBBO followed by immediate IA infusion of ⁸⁹ZrBVDFO and C—IV infusion of ⁸⁹ZrBVDFO, followed by BBBO 10 min after infusion was completed, as indicated by arrow in D panel, showing the highest accumulation of radioactivity in ipsilateral hemisphere upon BBBO/IA, D—curves demonstrating dynamics of ⁸⁹ZrBVDFO uptake in ipsilateral hemisphere upon IA/BBBI (blue line), BBBO/IA (red line) and IV/BBBO (gray line) indicating faster and higher uptake of ⁸⁹ZrBVDFO in animals treated with BBBO and IA infusion, E—increase of radioactivity in heart in the same groups (n=4), dynamic PET scans in IV/BBBO group was carried out for 45 min however no increase of radioactivity uptake was observed, NS—statistically nonsignificant, *—statistically significant difference.

FIG. 3 (includes FIGS. 3A and 3B) shows distribution of ⁸⁹ZrBVDFO in the brain. Representative coronal, sagittal and transverse PET images with overlaid mouse brain template obtained using PMOD 3.4 for mice scanned 1 h after: A—IA infusion of ⁸⁹ZrBVDFO with BBBI, B—BBBO followed by immediate IA infusion of 89ZrBVDFO and C—IV infusion of ⁸⁹ZrBVDFO, followed by BBBO 10 min after infusion was completed, demonstrating significantly higher brain uptake of ⁸⁹ZrBVDFO in BBBO/IA group compared to IA/BBBI and IV/BBBO groups with its major accumulation in right striatum, hippocampus and amygdala, * statistically significant difference.

FIG. 4 (includes FIGS. 4A-D) shows ⁸⁹ZrBVDFO delivery to the brain with and without BBBO and its biodistribution. Representative whole body volume rendered PET-CT images recorded 1 h and 24 h post infusion of ˜8.5 MBq (˜230 μCi) of ⁸⁹ZrBVDFO, demonstrating its biodistribution upon: A—IA infusion of ⁸⁹ZrBVDFO with BBBI, B—BBBO followed by immediate IA infusion of ⁸⁹ZrBVDFO and C—IV infusion of ⁸⁹ZrBVDFO, followed by BBBO 10 min after infusion was completed, D—PET based quantification of ⁸⁹ZrBVDFO uptake in ipsilateral hemisphere. E—Ex vivo biodistribution of ⁸⁹ZrBVDFO at 24 h after infusion in the same groups, showing in agreement with imaging higher uptake of ⁸⁹ZrBVDFO in ipsilateral hemisphere compared to contralateral hemisphere in BBBO/IA group and its higher brain accumulation in comparison with IA/BBBI and IV/BBBO.

FIG. 5 Conjugation of nanobody with DFO and radiolabeling with ⁸⁹Zr.

FIG. 6 (includes FIGS. 6A and 6D) PET imaging and dynamics of [⁸⁹Zr]NB(DFO)₂ uptake in ipsilateral hemisphere. Representative axial, sagittal and coronal PET images recorded 1 h after injection, illustrating brain uptake of ⁸⁹ZrNB(DFO)₂ upon: A—OBBBO followed by immediate IA infusion of 8.5 MBq of 89ZrNB(DFO)₂ reconstituted in 1 mL of saline at 0.15 mL/min, B—IA infusion with BBBI and C—IV infusion followed by BBBO at the 5 min after infusion was completed, showing the highest accumulation of radioactivity in ipsilateral hemisphere upon BBBO/IA, D—curves demonstrating dynamics of ⁸⁹ZrNB(DFO)₂ uptake in the ipsilateral hemisphere upon OBBBO/IA (red line), IA/BBBI (blue line), and IV/BBBO (grayline, arrow shows time of OBBBO) indicating highest uptake of ⁸⁹ZrNB(DFO)₂ in animals treated with OBBBO and IA infusion, each time point is presented as mean and SEM, n=4

FIG. 7 (includes FIGS. 7A and 7D) PET-CT imaging and ex vivo biodistribution of ⁸⁹ZrNB(DFO)₂ at 24 h after infusion. Whole body volume rendered PET-CT images recorded 1 h and 24 h post infusion of ˜8.5 MBq (˜230 μCi) of ⁸⁹ZrNB(DFO)₂, demonstrating its biodistribution upon: A—OBBBO followed by immediate IA infusion, B—IA infusion with BBBI and C—IV infusion followed by OBBBO 5 min after infusion was completed. D—Ex vivo biodistribution of ⁸⁹ZrNB(DFO)₂ at 24 h after infusion in the same groups (insert—PETbased quantification of ⁸⁹ZrNB(DFO)₂ uptake in ipsilateral hemisphere), showing in agreement with PET imaging higher uptake of ⁸⁹ZrNB(DFO)₂ in ipsilateral hemisphere compared to contralateral hemisphere in BBBO/IA group and its higher brain accumulation in comparison with IA/OBBBI and IV/OBBBO cohorts

FIG. 8 Conjugation of G₄(NH₂)₆₄ dendrimer with DFO, followed by capping of primary amines with butane-1,2-diol moieties and radiolabeling with ⁸⁹Zr.

FIG. 9 (includes FIGS. 9A and 9C) Time activity curves of ⁸⁹ZrG₄(DFO)₃(BFO)₁₁₀ uptake in ipsilateral hemisphere and corresponding PET imaging. A—Curves demonstrating dynamics of ⁸⁹ZrG₄(DFO)₃(Bdiol)₁₁₀ accumulation in the ipsilateral hemisphere upon OBBBO/IA (red line), IA/BBBI (blue line), and IV/BBBO (gray line, arrow shows when BBB was opened) indicating significantly lower uptake compared to nanobody and no benefits of OBBBO application, each time point is presented as mean and SEM, n=4; B—Representative orthogonal PET images obtained by summing frames between 5 and 10 min acquired during 30 min long dynamic scans; C—Representative axial PET images with scales adjusted to demonstrate whole body distribution of radioactivity (left panel) and absence of ⁸⁹ZrG4(DFO)₃(Bdiol)₁₁₀, in the brain (right panel) 1 h after infusion. Results demonstrate negligible retention of ⁸⁹ZrG₄(DFO)₃(Bdiol)₁₁₀ in the brain regardless BBB status and route of administration.

FIG. 10 (includes FIGS. 10A and 10D) 10 PET-CT imaging and ex vivo biodistribution of ⁸⁹ZrG4(DFO)₃(Bdiol)₁₁₀. A, B, C—representative whole body volume rendered PET-CT images recorded 1 h and 24 h post infusion of ⁸⁹ZrG4(DFO)₃(Bdiol)₁₁₀ for OBBBO/AI, AI/BBBI and IV/OBBBO infusions; D—ex vivo biodistribution of ⁸⁹ZrG4(DFO)₃(Bdiol)₁₁₀ at 24 h after infusion in the same mice (insert—scale was adjusted to show brain accumulation of ⁸⁹ZrG4(DFO)₃(Bdiol)₁₁₀, indication lack of ⁸⁹ZrG4(DFO)₃(Bdiol)₁₁₀ retention on the brain regardless method of administration and its renal clearance with minor hepatic uptake.

FIG. 11 (includes FIGS. 11a, 11b and 11c). The variability of cortical involvement during contrast agent infusion via ICA. (a, b) Representative T2* images during infusion of a contrast agent at a rate of 0.15 ml/min wherein the cortex was (a) or was not (b) perfused. (c) The constituent ratio of these phenomena.

FIG. 12 (includes FIGS. 12a and 12b). Use of real-time MRI to visualize the effect of cCCA closure on cortical trans-catheter perfusion. (a) Representative T2* images before (0s), 20 s, 60 s and 120 s after infusion of Gd at the rate of 0.15 ml/min. (b) Dynamic signal changes for two ROIs marked in (a). Graph lines and ROIs are shown in corresponding colors. Start represents the beginning of IA Gd infusion. Stop represents the end of the infusion.

FIG. 13 (includes FIG. 13(a) through 13(i)) Real-time MRI for predictable BBBO with histological validation. (a,d) Representative T2* images of Gd—CP. (b) Histogram analysis of pixel intensities in (a), showing two Gaussian distributions (red lines). Blue arrow points to where a cut-off of −62.02% was applied to separate the two distributions. (c) Segmented map shows the area where the relative signal change was smaller than −62.02%. (d) GD-CE map, (e) histogram analysis, and (f) segmented map (ΔS %>60%) right after mannitol infusion ended. (g) Scatter graph and (h) correlation analysis of the BBBO territory predicted by Gd—CP and assessed using Gd-CE (n=4). (i) The histological analyses show the region with extravasation of Evans blue.

FIG. 14 (includes FIG. 14(a) through Figure (c)) MRI and histological assessment post-BBBO. (a) 3 and 7 days after BBBO, T2-w and T2* w images did not indicate brain damage. No Gd enhancement in T1 images was observed in the brain, revealing that the BBB was resealed. Fluorescent staining of the BBBO region with GFAP (b) and IBA1 (c) showed comparable intensity between the ipsilateral and the contralateral hemisphere (3 ROIs/hemisphere as represented in lower magnification), indicating no inflammation after BBBO. Scale bar=100 μm.

FIG. 15 (includes FIGS. 15(a) through 15e). Visualization of cortical perfusion in epifluorescence microscopy. (a) The cranial window for microscopic imaging. (b) Representative fluorescent images show the perfusion territory of rhodamine without cCCA closure. (c) Dynamic signal changes of the ROI (circle) marked in (b). (d) Representative fluorescent images show the change of perfusion territory in the cortex pre- and post-cCCA closure. (e) Dynamic signal changes of the ROI (square) marked in (d). Start represents the beginning of rhodamine infusion. ON represents the weight is put on. Stop represents the end of the infusion.

FIG. 16 (includes FIG. 16(a) and FIG. 16(b)). Intravital 2PM visualization of cortical BBBO and drug extravasation. (a) Representative 2PM images showed the vessels permeability to rhodamine and bevacizumab. The arrow points to where BBB disruption started. (b) Quantitative measurement of fluorescent signal intensities in the selected extravascular regions marked in (a) over 15 min long dynamic imaging. The data was presented as mean±SEM from 7 ROIs. The grey shading indicated the IA infusion periods. Scale bar=50 μm.

FIG. 17 (includes FIG. 17(a) through FIG. 17(e)). Histological assessment of BV biodistribution and extravasation. (a, b) Coronal fluorescent photomicrographs of mouse cerebral cortex showed the distribution of infused BV-FITC in animals with BBBI and BBBO. (c, d) Quantification of fluorescence intensity of BV-FITC between the ipsilateral and contralateral hemisphere. (e) The ipsi-/contralateral ratio values were higher when the BBB was opened compared to that in animals with BBBI. Measurements are sampled from 3 ROIs/hemisphere as represented in lower magnification. Scale bar=50 μm.

### DETAILED DESCRIPTION

As discussed, we have now shown that infusion parameters can be adjusted based on feedback from real-time imaging and quantitative assessment of brain uptake of infused therapeutic molecules based on the imaging.

We discovered that administered therapeutic agents can be reliably assessed after administration though the blood-brain barrier of a subject.

In certain aspects, methods are provided that include (a) positioning a subject with a magnetic resonance (MR) image scanner; (b) disrupting the blood-brain barrier at an isolated region by administering in combination an effective amount of a blood-brain barrier opening agent and a contrast agent at the region; (c) acquiring MR images or optical images during the administering of above mentioned combination of agents; (d) administering one or more therapeutic agents through the blood-brain barrier with dynamic assessment of drug biodistribution based on PET imaging or optical imaging; and (e) imaging the subject to assess effects of the administered therapeutic agent(s). The assessment may include determination of uptake and/or clearance (including in brain or other targeted tissue) of the administered therapeutic agent(s). Administration of the one or more therapeutic agents also may be modified based on the assessment, for example infusion rates or dosages of the therapeutic agent(s) may be modified based on the assessment. The imaging suitably may be positron emission tomography (PET) imaging. The imaging also suitably may be optical imaging alone or in conjunction with another imaging technique such as optical imaging.

Suitable blood-brain barrier opening agents may suitably include but not limited to hyperosmolar agents as one or more mannitol, glycerin, isosorbide, or urea. The contrast agent suitably may be but not limited to gadolinium and/or Feraheme or a combination thereof, or an agent selected from the group consisting of: gadoterate (Dotarem); gadodiamide (Omniscan); gadobenate (MultiHance); gadopentetate (Magnevist, Magnegita, Gado-MRT ratiopharm); gadoteridol (ProHance); gadoversetamide (OptiMARK); gadoxetate (Primovist); gadobutrol (Gadovist); gadoterate (Dotarem); gadodiamide (Omniscan); gadobenate (MultiHance); gadopentetate (Magnevist); gadoteridol (ProHance); gadofosveset (Ablavar, formerly Vasovist); gadoversetamide (OptiMARK); gadoxetate (Eovist); and gadobutrol (Gadavist), or any photon-producing molceules such as green fluorescent protein (GFP) or red fluorescent protein (RFP) or others. Such labelled or photon-producing therapeutic molecules are particularly suitable for use with optical imaging as disclosed herein.

In certain embodiments, the isolated region of the blood-brain barrier is middle cerebral artery or basilar artery.

In certain embodiments, the invention also relates to any and all necessary catheter-related control equipment, pumps, drive systems, electrical and fluid control systems, as well as other separate or integrated systems for measuring and visualizing the method of the invention, e.g., fluoroscopic or other visualization systems, vital sign monitoring systems, and the like.

### Definitions

Unless defined otherwise, all technical and scientific terms used herein have the meaning commonly understood by a person skilled in the art to which this invention belongs. The following references, the entire disclosures of which are incorporated herein by reference, provide one of skill with a general definition of many of the terms (unless defined otherwise herein) used in this invention: Singleton et al., Dictionary of Microbiology and Molecular Biology (2^{nd} ed. 1994); The Cambridge Dictionary of Science and Technology (Walker ed., 1988); The Glossary of Genetics, 5th Ed., R. Rieger et al. (eds.), Springer Verlag (1991); and Hale & Marham, the Harper Collins Dictionary of Biology (1991). Generally, the procedures of molecular biology methods described or inherent herein and the like are common methods used in the art. Such standard techniques can be found in reference manuals such as for example Sambrook et al., (2000, Molecular Cloning—A Laboratory Manual, Third Edition, Cold Spring Harbor Laboratories); and Ausubel et al., (1994, Current Protocols in Molecular Biology, John Wiley & Sons, New-York).

The following terms may have meanings ascribed to them below, unless specified otherwise. However, it should be understood that other meanings that are known or understood by those having ordinary skill in the art are also possible, and within the scope of the present invention. All publications, patent applications, patents, and other references mentioned herein are incorporated by reference in their entirety. In the case of conflict, the present specification, including definitions, will control. In addition, the materials, methods, and examples are illustrative only and not intended to be limiting.

As used herein, the singular forms “a”, “and”, and “the” include plural references unless the context clearly dictates otherwise. All technical and scientific terms used herein have the same meaning.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The terms “disorders”, “diseases”, and “abnormal state” are used inclusively and refer to any deviation from the normal structure or function of any part, organ, or system of the body (or any combination thereof). A specific disease is manifested by characteristic symptoms and signs, including biological, chemical, and physical changes, and is often associated with a variety of other factors including, but not limited to, demographic, environmental, employment, genetic, and medically historical factors. Certain characteristic signs, symptoms, and related factors can be quantitated through a variety of methods to yield important diagnostic information. As used herein the disorder, disease, or abnormal state can be a cancer of the brain or a benign or malignant brain tumor. The disorder, disease, or abnormal state can also be a neurological disorder. As used herein, a neurological disorder is any disorder of the body's nervous system. Structural, biochemical or electrical abnormalities in the brain, spinal cord or other nerves can result in a range of symptoms. Examples of symptoms include paralysis, muscle weakness, poor coordination, loss of sensation, seizures, confusion, pain and altered levels of consciousness. There are many recognized neurological disorders, some relatively common, but many rare. They may be assessed by neurological examination, and studied and treated within the specialties of neurology and clinical neuropsychology. The term neurological disorder may also refer to any cancer arising from or within a neurological tissue, including brain cancer or tumors.

Neurological disorders can be categorized according to the primary location affected, the primary type of dysfunction involved, or the primary type of cause. The broadest division is between central nervous system (CNS) disorders and peripheral nervous system (PNS) disorders. The Merck Manual lists brain, spinal cord and nerve disorders in the following overlapping categories, all of which are contemplated by the invention:

Brain damage according to cerebral lobe, i.e., Frontal lobe damage, Parietal lobe damage, Temporal lobe damage, and Occipital lobe damage;

Brain dysfunction according to type: Aphasia (language), Dysarthria (speech), Apraxia (patterns or sequences of movements), Agnosia (identifying things/people), and Amnesia (memory);

Spinal cord disorders;

Peripheral neuropathy & other peripheral nervous system disorders;

Cranial nerve disorders such as Trigeminal neuralgia;

Autonomic nervous system disorders, such as dysautonomia and Multiple System Atrophy;

Seizure disorders, such as epilepsy;

Movement disorders of the central & peripheral nervous system, such as Parkinson's disease, essential tremor, amyotrophic lateral sclerosis (ALS), Tourette's Syndrome, multiple sclerosis & various types of peripheral neuropathy;

Sleep disorders, such as narcolepsy;

Migraines and other types of headache, such as cluster headache and tension headache;

Lower back and neck pain;

Central Neuropathy (see Neuropathic pain); and

Neuropsychiatric illnesses (diseases and/or disorders with psychiatric features associated with known nervous system injury, underdevelopment, biochemical, anatomical, or electrical malfunction, and/or disease pathology e.g., Attention deficit hyperactivity disorder, Autism, Tourette's Syndrome & some cases of Obsessive compulsive disorder as well as the neurobehavioral associated symptoms of degeneratives of the nervous system such as Parkinson's disease, Essential tremor, Huntington's disease, Alzheimer's disease, Multiple sclerosis & organic psychosis.)

As used herein, the term “obtaining” is understood herein as manufacturing, purchasing, or otherwise coming into possession of.

As used herein, “one or more” is understood as each value 1, 2, 3, 4, 5, 6, 7, 8, 9, 10, and any value greater than 10.

The term “or” is used inclusively herein to mean, and is used interchangeably with, the term “and/or,” unless context clearly indicates otherwise. For example, as used herein, filamin B or LY9 is understood to include filamin B alone, LY9 alone, and the combination of filamin B and LY9.

As used herein, “patient” or “subject” can mean either a human or non-human animal, preferably a mammal. By “subject” is meant any animal, including horses, dogs, cats, pigs, goats, rabbits, hamsters, monkeys, guinea pigs, rats, mice, lizards, snakes, sheep, cattle, fish, and birds. A human subject may be referred to as a patient.

The term “therapeutic effect” refers to a local or systemic effect in animals, particularly mammals, and more particularly humans caused by a pharmacologically or biologically active substance. The term thus means any substance intended for use in the diagnosis, cure, mitigation, treatment, or prevention of disease, or in the enhancement of desirable physical or mental development and conditions in an animal or human. A therapeutic effect can be understood as a decrease in tumor growth, decrease in tumor growth rate, stabilization or decrease in tumor burden, stabilization or reduction in tumor size, stabilization or decrease in tumor malignancy, increase in tumor apoptosis, and/or a decrease in tumor angiogenesis.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. As discussed, in the methods and compositions disclosed herein, a combination of one or more BBB opening agents and one or more contrast agents as a mixture or as an infusion of them in sequential manner or in combination are provided. The use of the term “in combination” does not restrict the order in which the therapies or agents (e.g., a contrast agent and a blood-brain barrier opening agent) are administered to a subject. Thus, for instance, a contrast agent can be administered prior to (e.g., 15 seconds, 0.5 minutes, 1 minute, 2 minutes, 3 minutes, 4 minutes, or 5 minutes or more), concomitantly with (e.g. contrast agent and blood-brain barrier opening agent administered as a combined composition, or contrast agent and hyperosmolar agent administered at substantially the same time such as sequential infusion, or subsequent to (e.g., 15 seconds, 0.5 minutes, 1 minute, 2 minutes, 3 minutes, 4 minutes, or 5 minutes or more) the administration of one or more blood-brain barrier opening agents.

As used herein, “therapeutically effective amount” means the amount of a compound that, when administered to a patient for treating a disease, is sufficient to effect such treatment for the disease, e.g., the amount of such a substance that produces some desired local or systemic effect at a reasonable benefit/risk ratio applicable to any treatment, e.g., is sufficient to ameliorate at least one sign or symptom of the disease, e.g., to prevent progression of the disease or condition, e.g., prevent tumor growth, decrease tumor size, induce tumor cell apoptosis, reduce tumor angiogenesis, prevent metastasis. When administered for preventing a disease, the amount is sufficient to avoid or delay onset of the disease. The “therapeutically effective amount” will vary depending on the compound, its therapeutic index, solubility, the disease and its severity and the age, weight, etc., of the patient to be treated, and the like. For example, certain compounds discovered by the methods of the present invention may be administered in a sufficient amount to produce a reasonable benefit/risk ratio applicable to such treatment. Administration of a therapeutically effective amount of a compound may require the administration of more than one dose of the compound.

As used herein, “treatment,” particularly “active treatment,” refers to performing an intervention to treat brain cancer in a subject, e.g., reduce at least one of the growth rate, reduction of tumor burden, reduce or maintain the tumor size, or the malignancy (e.g., likelihood of metastasis) of the tumor; or to increase apoptosis in the tumor by one or more of administration of a therapeutic agent, e.g., chemotherapy or hormone therapy; administration of radiation therapy (e.g., pellet implantation, brachytherapy), or surgical resection of the tumor, or any combination thereof appropriate for treatment of the subject based on grade and stage of the tumor and other routine considerations. Active treatment is distinguished from “watchful waiting” (i.e., not active treatment) in which the subject and tumor are monitored, but no interventions are performed to affect the tumor.

As used herein, “contrast agents” are a group of contrast media used to improve the visibility of internal body structures in but not limited to magnetic resonance imaging (MRI). The most commonly used compounds for contrast enhancement are gadolinium-based. MRI contrast agents alter the relaxation times of atoms within body tissues where they are present after oral or intravenous administration. In MRI scanners, sections of the body are exposed to a very strong magnetic field, then a radiofrequency pulse is applied causing some atoms (including those in contrast agents) to spin and then relax after the pulse stops. This relaxation emits energy which is detected by the scanner and is mathematically converted into an image. The MRI image can be weighted in different ways giving a higher or lower signal.

As used herein, the “brain” or “brain parenchyma” refers to the brain and brain stem tissues and any anatomic feature therein, and can include any anatomical region of the brain, such as the cerebrum (composed of the cortex and the corpus callosum), the diencephalon (composed of the thalamus, pineal body, and the hypothalamus), the brain stem (composed of the midbrain, pons, medulla oblongata), and the cerebellum. The brain or brain parenchyma can also include any functional region of the brain, including the frontal lobe, temporal lobe, central sulcus, parietal lobe, and occipital lobe, as well as deep structures of the limbic system, including the limbic lobe, corpus callosum, mammillary body, olfactory bulb, septal nuclei, amygdala, hippocampus, cingulate gyrus, fornix, and thalamus. The term “brain parenchyma” particularly refers to the functional portion of the brain, as compared to features that are merely structural.

As used herein, the term “compromised,” as in a compromised blood-brain barrier (BBB) refers to a BBB which has been partially, but reversibly disrupted. The term particularly refers to where the tight junctions between capillary endothelial cells of the BBB have been compromised such that molecules and components of the blood and CFS may pass or diffuse into the brain parenchym through the compromised tight junctions.

As used herein, the “blood-brain barrier” (BBB) refers to a highly selective permeability barrier that separates the circulating blood from the brain extracellular fluid (BECF) in the central nervous system (CNS). The blood-brain barrier is formed by capillary endothelial cells, which are connected by tight junctions with an extremely high electrical resistance of at least 0.1 Ωm. The blood-brain barrier allows the passage of water, some gases, and lipid soluble molecules by passive diffusion, as well as the selective transport of molecules such as glucose and amino acids that are crucial to neural function. On the other hand, the blood-brain barrier may prevent the entry of lipophilic, potential neurotoxins by way of an active transport mechanism of efflux mediated by P-glycoprotein. Astrocytes are also necessary to create the blood-brain barrier. A small number of regions in the brain, including the circumventricular organs (CVOs), do not have a blood-brain barrier. The blood-brain barrier occurs along all capillaries associated with cranial arteries and consists of tight junctions around the capillaries that do not exist in normal circulation. Endothelial cells restrict the diffusion of microscopic objects (e.g., bacteria) and large or hydrophilic molecules into the cerebrospinal fluid (CSF), while allowing the diffusion of small hydrophobic molecules. Cells of the barrier actively transport metabolic products such as glucose across the barrier with specific proteins. This barrier also includes a thick basement membrane and astrocytic endfeet.

Any compositions or methods provided herein can be combined with one or more of any of the other compositions and methods provided herein.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

In one aspect, methods include administering a therapeutic agent directly to the brain parenchyma through a compromised region of the blood-brain barrier in a subject having a brain disorder, comprising: (1) disrupting the blood-brain barrier (BBB) at an isolated region by locally administering an effective amount of a BBB opening agent at said region using a catheter, (2) administering a therapeutically effective amount of a therapeutic agent, wherein said disrupting step is performed using non-invasive MR (magnetic resonance) imaging with a contrast agent to visualize local parenchymal transcatheter perfusion at said isolated BBB region thereby indicating that the BBB region is compromised. As discussed, a contrast agent and blood-brain barrier opening agent may be administered in combination or sequentially to enable visualization of the location and formation of the disrupting of the blood-brain barrier.

In this embodiment, the first general step of the claimed method is to disrupt the BBB at a specific, local arterial region/territory by catheter-based administration of a blood-brain barrier opening agent (e.g., hyperosmolar agent such as mannitol) while using real-time MRI to visualize the detection of selective local parenchymal perfusion at the catheter tip, which shall indicate local disruption of the BBB (aka focal BBB disruption or BBBD).

Once the BBBD has been detected, a therapeutic agent may be administered by intraarterial infusion, e.g., through the same or separate catheter, at the site or proximal the site of BBBD or it can be administered systemically.

The subject then may be imaged to assess the administered therapeutic agent, for example, the uptake or clearance of the therapeutic by the subject.

In a particular embodiment, the infusion rate or injection rate of the blood-brain barrier opening agent (e.g. hyperosmolar agent such as mannitol) may be optimized prior to delivering a therapeutic agent in order to determine the optimized degree or level of selective perfusion of the brain parenchyma, i.e., which in turn reflects the degree of the BBBD or opening of the BBB. Exemplary rates of perfusion can include any suitable perfusion rate, such as, 0.01 ml/sec. The infusion rate can also include any range from about 0.001 ml/sec, to about 0.005 ml/sec, to about 0.01 ml/sec, to about 0.015 ml/sec, to about 0.02 ml/sec, to about 0.025 ml/sec, to about 0.03 ml/sec, to about 0.035 ml/sec, to about 0.04 ml/sec, to about 0.045 ml/sec, to about 0.05 ml/sec, to about 0.06 ml/sec, to about 0.07 ml/sec, to about 0.08 ml/sec, to about 0.09 ml/sec, to about 0.10 ml/sec, to about 0.20 ml/sec, to about 0.30 ml/sec, to about 0.40 ml/sec, to about 0.50 ml/sec, to about 0.60 ml/sec, to about 0.70 ml/sec, to about 0.80 ml/sec, to about 0.90 ml/sec, or more. In addition, the length of time of perfusion may be adjusted such that the degree of perfusion of the brain parenchym is optimized, and in turn, the degree of opening of the BBB. For example, perfusion may continuously or discontinuously operate for about 0.1 sec, about 0.2 sec, about 0.3 sec, about 0.4 sec, about 0.5 sec, about 0.6 sec, about 0.7 sec, about 0.8 sec, about 0.9 sec, about 1.0 sec, about 1-1.5 sec, to about 1.25-1.75 sec, to about 1.5-2.0 sec, to about 1.75-3.0 sec, to about 2.0-10.0 sec, to about 5.0-30.0 sec, to about 10.0-50.0 sec, to about 20.0-60.0 sec, to about 1-2 min, to about 2-5 min to about 5-10 min, to about 9-25 min, to about 24-50 min, to about 49-150 min, to up to several hours or more. When optimizing the degree of BBB opening, one of ordinary skill in the art may also take into account the other physical properties of the desired therapeutic agent to be delivered across the BBBD, including, for example, the molecular weight or size of the agent, the degree of lipophilicity of the agent, the presence of charge, and the concentration of the agent as delivered, and any other similar physical properties.

In still other embodiments, the placement of the tip of the perfusion catheter in the cranial artery (e.g. in the Basilar artery) may be adjusted and/or moved within the artery during MRI visualization to optimize the perfusion into the brain parenchymal, and thus, in turn, optimize the opening of the BBB. As discovered by the inventors, as opening of the BBB varies from subject to subject, and artery-to-artery it is preferable to optimize the opening of the BBB for infusion to each artery in each subject is desired to be treated by the methods of the invention.

### Treatable Disorders

The method of the invention may be used to treat any number of neurological disorders, including but not limited to brain cancer, neurodegenerative, neurological and psychiatric diseases.

Diseases can include neurological disorders, which can be categorized according to the primary location affected, the primary type of dysfunction involved, or the primary type of cause. The broadest division is between central nervous system (CNS) disorders and peripheral nervous system (PNS) disorders. The Merck Manual lists brain, spinal cord and nerve disorders in the following overlapping categories, all of which are contemplated by the invention:

Brain damage according to cerebral lobe, i.e., Frontal lobe damage, Parietal lobe damage, Temporal lobe damage, and Occipital lobe damage; Brain dysfunction according to type: Aphasia (language), Dysarthria (speech), Apraxia (patterns or sequences of movements), Agnosia (identifying things/people), and Amnesia (memory); Spinal cord disorders; Peripheral neuropathy & other peripheral nervous system disorders; Cranial nerve disorders such as Trigeminal neuralgia; Autonomic nervous system disorders, such as dysautonomia and Multiple System Atrophy; Seizure disorders, such as epilepsy; Movement disorders of the central and peripheral nervous system, such as Parkinson's disease, essential tremor, amyotrophic lateral sclerosis (ALS), Tourette's Syndrome, multiple sclerosis & various types of peripheral neuropathy; Sleep disorders, such as narcolepsy; Migraines and other types of headache, such as cluster headache and tension headache; Lower back and neck pain; Central Neuropathy (see Neuropathic pain); and Neuropsychiatric illnesses (diseases and/or disorders with psychiatric features associated with known nervous system injury, underdevelopment, biochemical, anatomical, or electrical malfunction, and/or disease pathology e.g., Attention deficit hyperactivity disorder, Autism, Tourette's Syndrome & some cases of Obsessive compulsive disorder as well as the neurobehavioral associated symptoms of degeneratives of the nervous system such as Parkinson's disease, Essential tremor, Huntington's disease, Alzheimer's disease, Multiple sclerosis & organic psychosis.)

Treatable diseases can also include brain tumors. Brain tumors are abnormal growths of new and unnecessary cells in or on the brain. It is thought that tumors occur when genetic factors or environmental damage impair normal cells so that they multiply and divide rapidly. There are many different kinds of brain tumors, which are classified in different ways depending on where the tumor originates, how quickly the tumor grows, and how destructive the tumor is.

Brain tumors are usually classified as either benign or malignant. Benign tumors tend to be slow-growing clusters of cells that rarely spread. Tumors are classified as malignant when they grow aggressively, invade other parts of the body, cause damage to critical functions, or are life threatening. Malignant tumors are also known as cancerous. Brain tumors that originate in the brain itself are called primary tumors. Primary brain tumors can start in the brain tissue, the brain lining (meninges), the skull, the nerves, or the pituitary gland. Tumors that originate somewhere else in the body and move into the brain are called metastatic tumors. Metastatic tumors are always malignant, since by definition they have invaded the brain from another part of the body. Very few primary brain tumors are benign, and even these tumors sometimes become malignant.

The invention contemplates treatment of all types and categories of brain tumors (whether cancerous or benign). Tumors can be optionally graded to indicate their degree of malignancy using a system developed by the World Health Organization (WHO). This system classifies tumors into four groups (WHO Grade I through IV) depending on factors such as how abnormal the cells are, how quickly the tumor is growing, the potential for invasion or spread of the tumor, and the blood supply of the tumor. Grade I tumors are considered benign and usually have very good survival rates. Grade II tumors are slow growing, but sometimes invade nearby tissue and/or recur after treatment. Grade III tumors have more abnormal cells and grow faster than Grade II tumors. Grade IV tumors are the most malignant. They grow rapidly and spread widely.

The invention contemplates treating any type of brain tumor, which can include the following types of benign brain tumors.

Meningiomas

A meningioma is a tumor that develops from the lining of the brain and spinal cord. It is the most common benign brain tumor in adults. A few meningiomas are malignant. The cause of meningiomas is unknown; however, some meningiomas are associated with specific genetic disorders, such as neurofibromatosis. Symptoms include seizure, headaches and loss of brain function (sensory problems, loss of coordination, etc.). Meningiomas usually grow slowly and may be treated at first with observation over time. For large meningiomas, surgery is usually the preferred treatment.

Acoustic Neuromas

Acoustic neuromas (a.k.a. vestibular schwannomas) are tumors arising from a cranial nerve. The tumor is usually benign and slow growing. The most common symptoms are hearing loss, ringing in the ears, vertigo (dizziness), and headaches. Options for treatment include observation, radiosurgery, and surgical resection. The ideal treatment in most cases is complete microsurgical tumor resection.

Pituitary Tumors

Pituitary tumors are tumors of the pituitary gland, which produces hormones to regulate the other glands in the body. These tumors may or may not secrete hormones. Often symptoms develop based on the type of hormone secreted. Some pituitary tumors are treated with medication alone, other with surgery, some with radiation, and some with a combination of all three treatments. Pituitary tumors represent approximately 10-15% of all brain tumors. They are most common in the third and fourth decade of life, and males and females are equally affected.

Colloid Cysts

Colloid cysts are benign tumors that only occur in the third ventricle, an area involved with cerebrospinal fluid flow. Tumors in this area can be life threatening by blocking the flow of cerebrospinal fluid, causing a condition called hydrocephalus. Hydrocephalus may cause headaches, nausea, vomiting, and even comas, which can lead to death. If the tumor is large enough, most neurosurgeons will treat the condition with surgical removal. Sometimes a ventricular shunt (a tube from the ventricles) is needed, which diverts and drains the cerebrospinal fluid and relieves pressure.

Arachnoid Cysts

An arachnoid cyst is a sac of cerebrospinal fluid that develops in the brain. Some of these cysts may develop in infancy, but often they are undiagnosed until a head injury occurs. Arachnoid cysts may cause no symptoms for a long time until they are large enough to put pressure on the brain or cause a deformity. Sometimes surgery is needed to create space around the cyst. Other cysts can be treated with a shunt.

Craniopharyngiomas

Craniopharyngiomas are benign tumors located above and behind the pituitary gland. These tumors grow slowly, but can cause vision problems or pituitary dysfunction. There is debate on how these tumors should be treated. Many neurosurgeons advocate surgical removal followed by radiation. In some cases, draining the cyst fluid may control the symptoms and halt growth.

Choroid Plexus Papillomas

Choroid plexus papillomas are benign tumors that occur in the brain's ventricular system from the cells that make spinal fluid. Treatment is usually surgical removal.

Hemangioblastomas

Hemangioblastomas are benign tumors of blood vessels that are often associated with cysts. They are usually treated with surgical removal, with or without radiation therapy.

Epidermoid and Dermoid Tumors

Epidermoid and dermoid tumors are benign tumors containing accumulated left over skin tissue within the head or spinal canal. The tumors usually require surgical removal.

The invention contemplates treating any type of brain tumor, which can include the following types of malignant brain tumors.

Primary Malignant Brain Tumors

The majority of primary brain tumors are malignant. Most primary malignant brain tumors arise from glial cells, which are tissues of the brain other than nerve cells or blood vessels. Unfortunately, these tumors can grow quickly and be very destructive. Management of these tumors depends primarily on the health of the patient and the location of the tumor. When feasible, treatment typically includes surgical removal followed by radiation and/or chemotherapy.

Metastatic Brain Tumors

These types of tumors originate in tissues outside of the brain, followed by metastasis to the brain. Metastatic tumors account for 10-15% of all brain tumors. The most common tumors that spread to the brain are those that originate in the lung, the breast, the kidney, or melanomas (skin cancer).

The method of the invention contemplates the treatment of any type of brain tumor by administration of therapeutically effective amounts of anti-cancer or anti-proliferative disorder agents. Such agents can include small molecule therapeutics, therapeutic peptides, therapeutic antibodies, and therapeutic nucleic acid molecules.

### Therapeutic Agents

The method of the invention contemplates the administration of any suitable therapeutic agent capable of treating a neurological disorder, including brain cancer.

Therapeutic agents can include any neurologically active agents acting at synaptic and neuroeffector junction sites. The neurologically active agent useful in the present invention may be one that acts at the synaptic and neuroeffector junctional sites; such as a cholinergic agonist, a anticholinesterase agent, catecholamine and other sympathomimetic drugs, an adrenergic receptor antagonist, an antimuscarinic drug, and an agent that act at the neuromuscular junction and autonomic ganglia.

Examples of suitable cholinergic agonists include, but are not limited to, choline chloride, acetylcholine chloride, methacholine chloride, carbachol chloride, bethanechol chloride, pilocarpine, muscarine, arecoline and the like. See Taylor, P., in The Pharmacological Basis of Therapeutics, Gilman, et al., eds., Pergamon Press, New York, 1990, 8th edition, Chapter 6, pp. 122-130.

Suitable anticholinesterase agents are exemplified by the group consisting of carbaril, physostigmine, neostigmine, edrophonium, pyridostigmine, demecarium, ambenonium, tetrahydroacridine and the like. See Taylor, P., in The Pharmacological Basis of Therapeutics, Gilman, et al., eds., Pergamon Press, New York, 1990, 8th edition, Chapter 7, pp. 131-149.

Suitable catecholamines and sympathomimetic drugs include the subclasses of endogenous catecholamines, beta-adrenergic agonists, alpha-adrenergic agonists and other miscellaneous adrenergic agonists.

Within the subclass of endogenous catecholamines, suitable examples include epinephrine, norepinephrine, dopamine and the like. Suitable examples within the subclass of beta-adrenergic agonists include, but are not limited to, isoproterenol, dobutamine, metaproterenol, terbutaline, albuterol, isoetharine, pirbuterol, bitolterol, ritodrine and the like. The subclass of .alpha.-adrenergic agonists can be exemplified by methoxamine, phenylephrine, mephentermine, metaraminol, clonidine, guanfacine, guanabenz, methyldopa and the like. Other miscellaneous adrenergic agents include, but are not limited to, amphetamine, methamphetamine, methylphenidate, pemoline, ephedrine and ethylnorepinephrine and the like. See Hoffman et al., in The Pharmacological Basis of Therapeutics, Gilman, et al., eds., Pergamon Press, New York, 1990, 8th edition, Chapter 10, pp. 187-220.

Adrenergic receptor antagonists include the subclasses of alpha-adrenergic receptor antagonists and beta-adrenergic receptor antagonists. Suitable examples of neurologically active agents that can be classified as alpha-adrenergic receptor antagonists include, but are not limited to, phenoxybenzamine and related haloalkylamines, phentolamine, tolazoline, prazosin and related drugs, ergot alkaloids and the like. Either selective or nonselective beta-adrenergic receptor antagonists are suitable for use in the present invention, as are other miscellaneous beta-adrenergic receptor antagonists. See Hoffman et al., in The Pharmacological Basis of Therapeutics, Gilman, et al., eds., Pergamon Press, New York, 1990, 8th edition, Chapter 11, pp. 221-243.

Antimuscarinic drugs are exemplified by the group consisting of atropine, scopolamine, homatropine, belladonna, methscopolamine, methantheline, propantheline, ipratropium, cyclopentolate, tropicamide, pirenzepine and the like. See Brown, J. H., in The Pharmacological Basis of Therapeutics, Gilman, et al., eds., Pergamon Press, New York, 1990, 8th edition, Chapter 8, pp. 150-165.

In addition, therapeutic agents that act at the neuromuscular junction and autonomic ganglia are contemplated by the invention. Suitable examples of such neurologically active agents that can be classified as agents that act at the neuromuscular junction and autonomic ganglia include, but are not limited to tubocurarine, alcuronium, beta-Erythroidine, pancuronium, gallamine, atracurium, decamethonium, succinylcholine, nicotine, labeline, tetramethylammonium, 1,1-dimethyl-4-phenylpiperazinium, hexamethonium, pentolinium, trimethaphan and mecamylamine, and the like. See Taylor, P., in The Pharmacological Basis of Therapeutics, Gilman, et al., eds., Pergamon Press, New York, 1990, 8th edition, Chapter 8, pp. 166-186.

The invention also contemplates the administration of drugs acting on the central nervous system and the peripheral nervous system. Such neurologically active agents can include nonpeptide neurotransmitters, peptide neurotransmitters and neurohormones, proteins associated with membranes of synaptic vessels, neuromodulators, neuromediators, sedative-hypnotics, antiepileptic therapeutic agents, therapeutic agents effective in the treatment of Parkinsonism and other movement disorders, opioid analgesics and antagonists and antipsychotic compounds.

Nonpeptide neurotransmitters include the subclasses of neutral amino acids—such as glycine and gamma-aminobutyric acid and acidic amino acids—such as glutamate, aspartate, and NMDA receptor antagonist-MK801 (Dizocilpine Maleate). L. L. Iversen, Neurotransmissions, Research biochemicals International, Vol. X, no. 1, February 1994. Other suitable nonpeptide neurotransmitters are exemplified by acetylcholine and the subclass of monoamines—such as dopamine, norepinephrine, 5-hydroxytryptamine, histamine, and epinephrine.

Neurotransmitters and neurohormones that are neuroactive peptides include the subclasses of hypothalamic-releasing hormones, neurohypophyseal hormones, pituitary peptides, invertebrate peptides, gastrointestinal peptides, those peptides found in the heart—such as atrial naturetic peptide, and other neuroactive peptides. See J. H. Schwartz, “Chemical Messengers: Small Molecules and Peptides” in Principles of Neural Science, 3rd Edition; E. R. Kandel et al., Eds.; Elsevier: New York; Chapter 14, pp. 213-224 (1991).

The subclass of hypothalamic releasing hormones includes as suitable examples, thyrotropin-releasing hormones, gonadotropin-releasing hormone, somatostatins, corticotropin-releasing hormone and growth hormone-releasing hormone.

The subclass of neurohypophyseal hormones is exemplified by agents such as vasopressin, oxytocin, and neurophysins. Likewise the subclass of pituitary peptides is exemplified by the group consisting of adrenocorticotropic hormone, beta-endorphin, alpha-melanocyte-stimulating hormone, prolactin, luteinizing hormone, growth hormone, and thyrotropin.

Suitable invertebrate peptides are exemplified by the group comprising FMRF amide, hydra head activator, proctolin, small cardiac peptides, myomodulins, buccolins, egg-laying hormone and bag cell peptides. The subclass of gastrointestinal peptides includes such therapeutic agents as vasoactive intestinal peptide, cholecystokinin, gastrin, neurotensin, methionine-enkephalin, leucine-enkephalin, insulin and insulin-like growth factors I and II, glucagon, peptide histidine isoleucineamide, bombesin, motilin and secretins.

Suitable examples of other neuroactive peptides include angiotensin II, bradykinin, dynorphin, opiocortins, sleep peptide(s), calcitonin, CGRP (calcitonin gene-related peptide), neuropeptide Y, neuropeptide Yy, galanin, substance K (neurokinin), physalaemin, Kassinin, uperolein, eledoisin and atrial naturetic peptide.

Proteins associated with membranes of synaptic vesicles include the subclasses of calcium-binding proteins and other synaptic vesicle proteins.

The subclass of calcium-binding proteins further includes the cytoskeleton-associated proteins—such as caldesmon, annexins, calelectrin (mammalian), calelectrin (torpedo), calpactin I, calpactin complex, calpactin II, endonexin I, endonexin II, protein II, synexin I; and enzyme modulators—such as p65.

Other synaptic vesicle proteins include inhibitors of mobilization (such as synapsin Ia,b and synapsin IIa,b), possible fusion proteins such as synaptophysin, and proteins of unknown function such as p29, VAMP-1,2 (synaptobrevin), VAT-1, rab 3A, and rab 3B. See J. H. Schwartz, “Synaptic Vessicles” in Principles of Neural Science, 3rd Edition; E. R. Kandel et al., Eds.; Elsevier: New York; Chapter 15, pp. 225-234(1991).

Neuromodulators can be exemplified by the group consisting of CO2 and ammonia (E. Flory, Fed. Proc., 26, 1164-1176 (1967)), steroids and steroid hormones (C. L. Coascogne et al., Science, 237, 1212-1215 (1987)), adenosine and other purines, and prostaglandins.

Neuromediators can be exemplified by the group consisting of cyclic AMP, cyclic GMP (F. E. Bloom, Rev. Physiol. Biochem. Pharmacol., 74, 1-103 (1975), and cyclic nucleotide-dependent protein phosphorylation reactions (P. Greengard, Distinguished Lecture Series of the Society of General Physiologists, 1, Raven Press: New York (1978)).

Sedative-hypnotics can be exemplified by the group consisting of benzodiazepines and buspirone, barbiturates, and miscellaneous sedative-hypnotics. A. J. Trevor and W. L. Way, “Sedative-Hypnotics” in Basic and Clinical Pharmacology; B. G. Katzung, Ed.; Appleton and Lange; Chapter 21, pp. 306-319 (1992).

Suitable antiepileptic drugs can be exemplified by the groups consisting of, but not limited to, hydantoins such as phenytoin, mephenytoin, and ethotoin; anticonvulsant barbiturates such as phenobarbital and mephobarbital; deoxybarbiturates such as primidone; iminostilbenes such as carbamazepine; succinimides such as ethosuximide, methsuximide, and phensuximide; valproic acid; oxazolidinediones such as trimethadione and paramethadione; benzodiazepines and other antiepileptic agents such as phenacemide, acetazolamide, and progabide. See T. W. Rallet al., “Drugs Effective in the Therapy of the Epilepsies”, in The Pharmacological Basis of Therapeutics, 8th Edition; A. G. Gilman et al., Eds.; Pergamon Press: New York; Chapter 19, pp. 436-462 (1990).

Neurologically active agents that are effective in the treatment of Parkinsonism and other movement disorders include, but are not limited to, dopamine, levodopa, carbidopa, amantadine, baclofen, diazepam, dantrolene, dopaminergic agonists such as apomorphine, ergolines such as bromocriptine, pergolide, and lisuride, and anticholinergic drugs such as benztropine mesylate, trihexyphenidyl hydrochloride, procyclidine hydrochloride, biperiden hydrochloride, ethopropazine hydrochloride, and diphenhydramine hydrochloride. See J. M. Cedarbaum et al., “Drugs for Parkinson's Disease, Spasticity, and Acute Muscle Spasms”, in The Pharmacological Basis of Therapeutics, 8th Edition; A. G. Gilman et al., Eds.; Pergamon Press: New York; Chapter 20, pp. 463-484 (1990).

Suitable opioid analgesics and antagonists can be exemplified by the group consisting of, but not limited to, endogenous opioid peptides such as enkephalins, endorphins, and dynorphins; morphine and related opioids such as levorphanol and congeners; meperidine and congeners such as piperidine, phenylpiperidine, diphenoxylate, loperamide, and fentanyl; methadone and congeners such as methadone and propoxyphene; pentazocine; nalbuphine; butorphanol; buprenorphine; meptazinol; opioid antagonists such as naloxone hydrochloride; and centrally active antitussive agents such as dextromethorphan. See J. H. Jaffe et al., “Opioid Analgesics and Antagonists” in The Pharmacological Basis of Therapeutics, 8th Edition; A. G. Gilman et al., Eds.; Pergamon Press: New York; Chapter 21, pp. 485-521 (1990)

Neurologically active agents that can be used to treat depression, anxiety or psychosis are also useful in the present conjugate. Suitable antipsychotic compounds include, but are not limited to, phenothiazines, thioxanthenes, dibenzodiazepines, butyrophenones, diphenylbutylpiperidines, indolones, and rauwolfia alkaloids. Mood alteration drugs that are suitable for use in the present invention include, but are not limited to, tricyclic antidepressants (which include tertiary amines and secondary amines), atypical antidepressants, and monoamine oxidase inhibitors. Examples of suitable drugs that are used in the treatment of anxiety include, but are not limited to, benzodiazepines. R. J. Baldessarini, “Drugs and the Treatment of Psychiatric Disorders”, in The Pharmacological Basis of Therapeutics, 8th Edition; A. G. Gilman et al., Eds.; Pergamon Press: New York; Chapter 18, pp. 383-435 (1990).

The neurologically active agent useful in the present conjugate may also be a neuroactive protein, such as human and chimeric mouse/human monoclonal antibodies, erythropoietin and G-CSF, orthoclone OKT3, interferon-gamma, interleukin-1 receptors, t-PA (tissue-type plasminogen activator), recombinant streptokinase, superoxide dismutase, tissue factor pathway inhibitor (TFPI). See Therapeutic Proteins: Pharmacokinetics and Pharmacodynamics; A. H. C. Kung et al., Eds.; W. H. Freeman: New York, pp 1-349 (1993).

The neurologically active agent useful in the present conjugate may also be a neuroactive nonprotein drug, such as neurotransmitter receptors and pharmacological targets in Alzheimer's disease; Design and Synthesis of BMY21502: A Potential Memory and Cognition Enhancing Agent; muscarinic agonists for the central nervous system; serotonic receptors, agents, and actions; thiazole-containing 5-hydroxytryptamine-3 receptor antagonists; acidic amino acids as probes of glutamate receptors and transporters; L-2-(carboxycyclopropyl)glycines; and N-Methyl-D-aspartic acid receptor antagonists. See Drug Design for Neuroscience; A. P. Kozikowski, Ed.; Raven Press: New York, pp 1-469 (1993).

The neurologically active agent useful in the present invention may also be an approved biotechnology drug or a biotechnology drug in development. Exemplary members of this group are included on Tables 1 and 2 of U.S. Pat. No. 5,604,198 (approved biotechnology drugs and biotechnology drugs in development, respectively) and may be found in J. E. Talmadge, Advanced Drug Delivery Reviews, 10, 247-299 (1993), each of which are incorporated by reference.

The invention also contemplates administration of cancer therapies through the BBBD. Non-limiting examples of anti-cancer agents and drugs that can be used in combination with one or more compositions and methods of the invention for the treatment of cancer include, but are not limited to, one or more of: 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, derivatives, enantiomers and/or functionally equivalent compositions thereof.

Other examples of agents useful in the treatment of cancer include, but are not limited to, one or more of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf r3, ior c5, BABS, anti-FLK-2, MDX-260, ANA Ab, SMART 1D10 Ab, SMART ABL 364 Ab and ImmuRAIT-CEA.

### Therapeutic Antibodies and Other Macromolecules and Biotechnolgical Drugs

The method of the invention specifically contemplates the enhanced ability to deliver therapeutic antibodies to a subject across the blood-brain barrier. The term “antibody” as used herein refers to immunoglobulin molecules and immunologically active portions of immunoglobulin (Ig) molecules, i.e., molecules that contain an antigen-binding site that specifically binds (immunoreacts with) an antigen, comprising at least one, and preferably two, heavy (H) chain variable regions (abbreviated herein as VH), and at least one and preferably two light (L) chain variable regions (abbreviated herein as VL). Such antibodies include, but are not limited to, polyclonal, monoclonal, chimeric, single chain, Fab, Fab′ and F(ab′)2 fragments, and an Fab expression library. The VH and VL regions can be further subdivided into regions of hypervariability, termed “complementarity determining regions” (“CDR”), interspersed with regions that are more conserved, termed “framework regions” (FR). The extent of the framework region and CDR's has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917, which are incorporated herein by reference). Each VH and VL is composed of three CDR's and four FRs, arranged from amino-terminus to carboxy-terminus in the following order: FR1, CDR1, FR2, CDR2, FR3, CDR3, FR4. In general, antibody molecules obtained from humans relates to any of the classes IgG, IgM, IgA, IgE and IgD, which differ from one another by the nature of the heavy chain present in the molecule. Certain classes have subclasses as well, such as IgG1, IgG2, and others. Furthermore, in humans, the light chain may be a kappa chain or a lambda chain. Reference herein to antibodies includes a reference to all such classes, subclasses and types of human antibody species.

Antibodies can be prepared from the intact polypeptide or fragments containing peptides of interest as the immunizing agent. A preferred antigenic polypeptide fragment is 15-100 contiguous amino acids of protein antigen of interest. In one embodiment, the peptide is located in a non-transmembrane domain of the polypeptide, e.g., in an extracellular or intracellular domain. An exemplary antibody or antibody fragment binds to an epitope that is accessible from the extracellular milieu and that alters the functionality of the protein. In certain embodiments, the present invention comprises antibodies that recognize and are specific for one or more epitopes of a protein antigen of interest.

The preparation of monoclonal antibodies is well known in the art; see for example, Harlow et al., Antibodies: A Laboratory Manual, page 726 (Cold Spring Harbor Pub. 1988). Monoclonal antibodies can be obtained by injecting mice or rabbits with a composition comprising an antigen, verifying the presence of antibody production by removing a serum sample, removing the spleen to obtain B lymphocytes, fusing the lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by techniques well known in the art.

In other embodiments, the antibody can be recombinantly produced, e.g., produced by phage display or by combinatorial methods. Phage display and combinatorial methods can be used to isolate recombinant antibodies that bind to a target disease peptide in the brain or fragments thereof (as described in, e.g., Ladner et al. U.S. Pat. No. 5,223,409; Fuchs et al. (1991) Bio/Technology 9:1370-1372; Hay et al. (1992) Hum Antibod Hybridomas 3:81-85; Huse et al. (1989) Science 246:1275-1281; Clackson et al. (1991) Nature 352:624-628; Gram et al. (1992) PNAS 89:3576-3580.

Human monoclonal antibodies can also be generated using transgenic mice carrying the human immunoglobulin genes rather than the mouse system. Splenocytes from these transgenic mice immunized with the antigen of interest are used to produce hybridomas that secrete human mAbs with specific affinities for epitopes from a human protein (see, e.g., Wood et al. International Application WO 91/00906; Lonberg, N. et al. 1994 Nature 368:856-859; Green, L. L. et al. 1994 Nature Genet. 7:13-21; Morrison, S. L. et al. 1994 Proc. Natl. Acad. Sci. USA 81:6851-6855).

A therapeutically useful antibody to the components of the complex of the invention or the complex itself may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found in Jones et al., Nature 321: 522, 1986 and Singer et al., J. Immunol. 150: 2844, 1993. The antibodies can also be derived from human antibody fragments isolated from a combinatorial immunoglobulin library; see, for example, Barbas et al., Methods: A Companion to Methods in Enzymology 2, 119, 1991. In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546, 1985. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies. Single chain antibodies are formed by linking the heavy and light chain fragments of the Fv region via an amino acid bridge, resulting in a single chain polypeptide. Antibody fragments that recognize specific epitopes, e.g., extracellular epitopes, can be generated by techniques well known in the art. Such fragments include Fab fragments produced by proteolytic digestion, and Fab fragments generated by reducing disulfide bridges. When used for immunotherapy, the monoclonal antibodies, fragments thereof, or both may be unlabelled or labeled with a therapeutic agent. These agents can be coupled directly or indirectly to the monoclonal antibody by techniques well known in the art, and include such agents as drugs, radioisotopes, lectins and toxins.

The dosage ranges for the administration of monoclonal antibodies are large enough to produce the desired effect, and will vary with age, condition, weight, sex, age and the extent of the condition to be treated, and can readily be determined by one skilled in the art. Dosages can be about 0.1 mg/kg to about 2000 mg/kg. The monoclonal antibodies can be administered intravenously, intraperitoneally, intramuscularly, and/or subcutaneously.

As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein. A protein of the invention, or a derivative, fragment, analog, homolog or ortholog thereof, may be utilized as an immunogen in the generation of antibodies that immunospecifically bind these protein components.

Fully human antibodies are also contemplated. Fully humanized antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol. 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology, 10:779-783 (1992)); Lonberg et al. (Nature, 368:856-859 (1994)); Morrison (Nature, 368:812-13 (1994)); Fishwild et al, (Nature Biotechnology, 14:845-51 (1996)); Neuberger (Nature Biotechnology, 14:826 (1996)); and Lonberg and Huszar (Intern. Rev. Immunol., 13:65-93 (1995)).

Human antibodies may additionally be produced using transgenic nonhuman animals which are modified so as to produce fully human antibodies rather than the animal's endogenous antibodies in response to challenge by an antigen. The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096.

Fab Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., Science 246:1275-1281 (1989)) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F(ab′)2 fragment produced by pepsin digestion of an antibody molecule; (ii) an Fab fragment generated by reducing the disulfide bridges of an F(ab′)2 fragment; (iii) an Fab fragment generated by the treatment of the antibody molecule with papain and a reducing agent and (iv) Fv fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit. Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. Similar procedures are disclosed in WO 93/08829, published May 13, 1993, and Traunecker et al., EMBO J., 10:3655-3659 (1991).

Antibody variable domains with the desired binding specificities (antibody-antigen combining sites) can be fused to immunoglobulin constant domain sequences. For further details of generating bispecific antibodies see, for example, Suresh et al., Methods in Enzymology, 121:210 (1986); and Brennan et al., Science 229:81 (1985).

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)2 molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The “diabody” technology described by Hollinger et al., Proc. Natl. Acad. Sci. USA 90:6444-6448 (1993) has provided an alternative mechanism for making bispecific antibody fragments. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991). Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA.

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Immunoconjugates

The invention also pertains to immunoconjugates comprising an antibody conjugated to a chemical agent, or a radioactive isotope (i.e., a radioconjugate) for administration to the brain using the methods of the invention. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257: 286-288 (1982) via a disulfide-interchange reaction.

A therapeutically effective amount of an antibody as disclosed herein relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 500 mg/kg body weight.-Common dosing frequencies may range, for example, from twice daily to once a week.

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York. The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions. The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

### Formulations

Preparations for administration of a therapeutic of the invention include sterile aqueous or non-aqueous solutions, suspensions, and emulsions, and in particular, formulations suitable for intraarticular infusion or injection via a catheter. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Vehicles include sodium chloride solution, Ringer's dextrose, dextrose and sodium chloride, lactated Ringer's intravenous vehicles including fluid and nutrient replenishers, electrolyte replenishers, and the like. Preservatives and other additives may be added such as, for example, antimicrobial agents, anti-oxidants, chelating agents and inert gases and the like.

The compounds, nucleic acid molecules, polypeptides, and antibodies (also referred to herein as “therapeutic agents”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, intraperitoneal, and rectal administration, and by intraarterial infusion via a catheter. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration (e.g., via a catheter system), suitable carriers include physiological saline, bacteriostatic water, Cremophor (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., the therapeutic complex of the invention) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethan-e, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system. The pharmaceutical compositions can be included in a container, pack, or dispenser together with instructions for administration.

A therapeutically effective dose refers to that amount of the therapeutic sufficient to result in amelioration or delay of symptoms. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Reference is also made to Zawadzki et al., BMJ Case Rep. 2019; 12:e014469; US Published Patent Application 20170079581; W Lesniak et al., J. Nucl. Med. 10:2967 (Oct. 12, 2018); and W. Lesniak et al., European Journal of Medicine and Molecular Imaging vol. 46, Issue 9, 1 Aug. 2019, Pages 1940-1951, incorporated by reference herein, for disclosure of procedures and systems useful in the present methods and systems.

### EXAMPLES

This invention is further illustrated by the following examples which should not be construed as limiting. Those skilled in the art will recognize that the invention may be practiced with variations on the disclosed structures, materials, compositions and methods, and such variations are regarded as within the ambit of the invention.

### Example 1

We used ⁸⁹ZrBVDFO and PET to capture dynamics of BV after IA delivery to the brain and compared its brain distribution with and without BBBO, and also compared IA and systemic (intravenous, IV) delivery under the same conditions.

### Materials and Methods

### Materials

All chemicals were purchased from Sigma-Aldrich (Milwaukee, Wis.) or Fisher Scientific

(Tewksbury, Mass.) unless otherwise specified. AVASTIN® (BV, Roche, 4 mL, 25 mg/mL) was obtained from Johns Hopkins Hospital Pharmacy. ⁸⁹Zr(C₂O₄)₂ (t_{1/2}=78.4 h) and 1-(4-isothiocyanatophenyl)-3-[6,17-dihydroxy-7,10,18,21-tetraoxo-27-(N-acetylhydroxylamino)-6,11,17, 22-tetraazaheptaeicosine] thiourea (p-SCN-Bn-DFO, Cat. #B-705) were obtained from Washington University (St. Louis, Mo.) and Macrocyclics (Plano, Tex.), respectively. All reagents and solvents were used as received without further purification.

### Study Design

In the first step, we conjugated BV with DFO chelator and characterized resulting conjugate by means of its molecular weight and binding to vascular endothelial growth factor (VEGF). After radiolabeling of BVDFO with ⁸⁹Zr we have evaluated its accumulation in brain using three groups of mice (n=4) treated with: I—IA infusion of ⁸⁹ZrBVDFO with intact BBB (abbreviated to IA/BBBI), II—IA infusion of ⁸⁹ZrBVDFO immediately after BBBO with 25% mannitol (abbreviated to BBBO/IA) and III—IV infusion of ⁸⁹ZrBVDFO and subsequent BBBO with mannitol at 15 minute interval, which allowed to assess the brain uptake of ⁸⁹ZrBVDFO prior and after BBBO in the same animals (abbreviated to IV/BBBO). Accumulation of radioactivity in the brain during and after infusions of ⁸⁹ZrBVDFO reconstituted in 1 mL of saline and delivered at 0.15 mL/min were monitored by (one-bed) dynamic PET over 0.5 h and subsequent whole-body (two-beds) PET-CT imaging. Next day PET-CT imaging was repeated and animals were sacrificed to perform ex vivo biodistribution of ⁸⁹ZrBVDFO.

### Synthesis of BVDFO

Avastin® (BV) is formulated in 240 mg of α,α-trehalose dehydrate, 23.2 mg sodium phosphate (monobasic, monohydrate), 4.8 mg sodium phosphate (bibasic, anhydrous), 1.6 mg polysorbate 20 and water thus for conjugation with DFO 15 mg of the antibody was purified using ultrafiltration with Millipore Amicon Ultra Centrifugal Filters 50K (cat #: VV-29969-76) and saline. After purification 10 mg of bevacizumab was reconstituted in 2 mL of saline, pH was adjusted to 9 with small amount of 0.1 M Na₂CO₃, five-fold molar equivalent of SCN-Bn-DFO dissolved in DMSO was added and conjugation was carried out for 30 min at 37° C. in a thermomixer at 550 r.p.m. Resulting BVDFO conjugate was purified as described above, reconstituted in saline at 10 mg/mL and 0.1 mL aliquots were kept at −20° C. until further use. The protein concertation in purified BV and BVDFO was determined by means of absorbance at 280 nm obtained by collecting UV-vis spectrum ranging from 200 to 750 nm and extinction coefficient of 1.52 cm×mL/mg derived from Beer's law and 280 nm absorbance of a 2.5 mg/mL solution of BV in PBS.

### Matrix-Assisted Laser Desorption Ionization-Time-of-Flight (MALDI-TOF)

To determine average number of DFO molecules conjugated with bevacizumab MALDITOFspectra of unmodified antibody and BVDFO conjugate were recorded on a Voyager DE-STR spectrophotometer, using 2,5-dihydroxybenzoic acid (DHB) as a matrix. First protein samples were desalted using Zeba™ spin columns 7K MWCO (cat. #89882, Thermo Fisher Scientific) and 10 μL of elutes were mixed with 10 μL of matrix (10 mg/mL). Then 1 μL of resulting mixture was placed on the target plate (in triplicate) and evaporated. Matrix was dissolved in 50% MeOH and 0.1% TFA aqueous solution. Number of shots and laser power was adjusted according to spectrum quality.

### Functional Binding Assay of BV and BVDFO to VEGF

The ELISA assay devoted for assessment of bevacizumab concentration has been used to assess the binding capacity of unmodified BV and BVDFO conjugate. The assay was carried out using Bevacizumab ELISA (ImmunoGuide, Eagle Bioscience) according to the manufacturer protocol. Briefly, 100 μg/mL, 50 μg/mL, 25 μg/mL of BV and BVDFO, as well as the provided standards were diluted 1:1000, and pipetted in 6 repetitions into the wells of the microtiter plate coated with recombinant human VEGF-A. Plate was incubated for 60 min in the room temperature and washed 3× with buffer. Next, horseradish peroxidase (HRP) conjugated anti-human IgG monoclonal antibody was added to each well, and incubated in room temperature for 30 min. Plate was washed 3× with the buffer and ready-to-use TMB substrate solution was added to each well. After 15 min incubation in dark, stop solution was added to each well and the color change from blue to yellow was observed. The absorbance at 450 nm was read using Victor 3 plate reader (Perkin Elmer) within 10 min after addition of the stop solution and expressed as optical density (OD).

### Radiolabeling of BVDFO

Radiolabeling of BVDFO with ⁸⁹Zr was performed using reported procedure with modifications (16). Concentration of the protein in an obtained ⁸⁹ZrBVDFO was determined based on absorbance at 280 nm from UV-Vis spectrum collected on a Nanodrop 2000 UV-vis spectrophotometer (Thermo Fisher Scientific) and area under peak in a SEC chromatogram recorded using absorbance at 280 nm. Size exclusion chromatography was carried out using a Varian ProStar pump, Phenomenex Yarra SEC-4000 column and 0.1 M phosphate buffer (pH 6.4) as a mobile phase at flow rate of 1 mL/min. Elution was monitored using a Varian ProStar UV absorbance detector set to 280 nm and a radioactive single-channel flow-through radiation detector (Bioscan model 105S). ⁸⁹ZrBVDFO was fabricated with 99.4% radiochemical purity and 81.4±7.4 MBq/mg (2.2±0.2 μCi/mg) specific activity. For further studies, ⁸⁹ZrBVDFO was diluted with sterile saline.

PET-CT Imaging of IA and IV Delivery of 89ZrBVDFO with or without BBBO

All animal procedures were carried out under protocols approved by the Johns Hopkins Animal Care and Use Committee. Under general anesthesia with 1-2% isoflurane a catheter was placed in internal carotid artery of C₃HeB/FeJ (Jackson, stock No. 000658), male, 6-8 weeks old mice, as we described previously (22) and animal was transferred to the PET-CT scanner. The BBB was opened with a minute-long infusion of 25% mannitol at a speed 0.15 mL/min. ˜8.5 MBq (˜230 μCi)⁸⁹ZrBVDFO reconstituted in 1 mL of saline was infused IA or IV over 5 minutes also at the speed of 0.15 mL/min. Accumulation of ⁸⁹ZrBVDFO in the brain was initially monitored with dynamic scans (for IA infusions 30 second frames in one bed position were collected for 30 min, for IV infusion 30 second frames in one bed position, collected in 45 min: 15 minutes before BBBO and 30 minutes after BBBO) followed by whole body PET/CT imaging acquired around 1 h post infusion (i.p.), in two bed positions and 7 min per bed on an ARGUS small-animal PET/CT scanner (Sedecal, Madrid, Spain). A CT scan (512 projections) was performed after dynamic scan for anatomical co-registration. PET/CT imaging was repeated around 24 h post infusion. PET data were reconstructed using the two-dimensional ordered subsets-expectation maximization algorithm (2D-OSEM) and corrected for dead time and radioactive decay. Presented whole body images were generated using Amira® (FEI, Hillsboro, Oreg.) and dynamic scans (brain and heart radioactivity accumulation) and radioactivity distribution in different brain regions were analyzed with PMOD 4.3 (PMOD Technologies LLC, Zurich, Switzerland).

### Ex Vivo Biodistribution of ⁸⁹ZrBVDFO

Upon completion of PET-CT at 24 h post infusion of ⁸⁹ZrBVDFO mice were sacrificed, blood, brain (divided into right and left hemispheres) and selected organs were harvested and weighed. The radioactivity in collected samples was measured on a PerkinElmer—2480 Automatic Gamma Counter. To calculate the percent injected dose per gram of tissue (% ID/g), triplicate radioactive standards (0.01% of the injected dose) were counted along with tissue samples. Biodistribution data shown is mean±the standard error of the mean (SEM).

### Statistical Analysis.

PROC MIXED (SAS 9.4) was used for statistical analysis, with the lowest means square (LMS) test for comparison between groups. The statements “repeated” and “random” were used for repeated measures and to express random effects, respectively.

### Results

### Radiolabeling

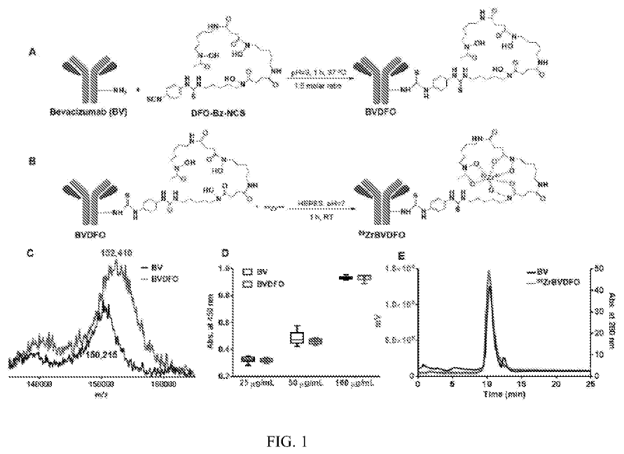

As depicted in FIG. 1A, radiolabeling of BV with zirconium-89 involved conjugation on average 3 molecules of DFO and subsequent chelation of ⁸⁹Zr⁴⁺. The average number of DFO molecules conjugated with BV was derived from the increase of molecular weight detected by MADLI-TOF spectrometry (FIG. 1 C). BV and BVDFO conjugate exhibited similar binding to VEGF as confirmed by ELISA (FIG. 1 D). Co-elution of ⁸⁹ZrBVDFO with intact BV observed in the SEC chromatogram confirmed radiolabeling of BVDFO (FIG. 1 E). ⁸⁹ZrBVDFO was prepared with 81.4±7.4 MBq/mg, 99±2% and 73±3% specific activity, radiochemical purity and efficiency, respectively.

### PET Imaging

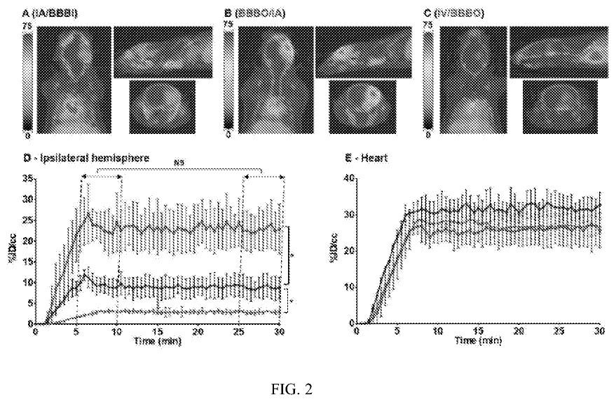

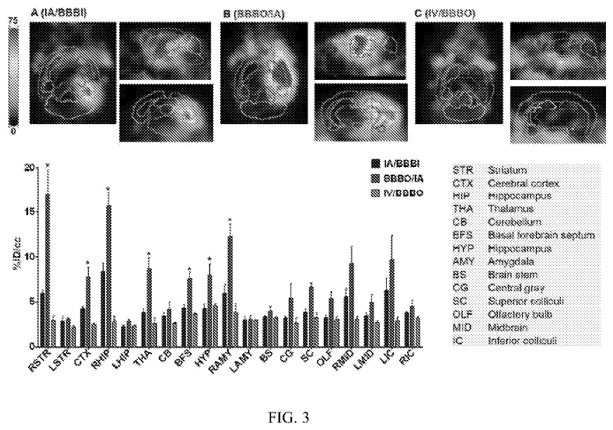

The IA delivery of ⁸⁹ZrBVDFO with BBBI resulted in a gradual accumulation of radioactivity during infusion in the ipsilateral hemisphere reaching 9.66±2.04% ID/cc between 1^{st} and 6th minute after infusion was completed and signal remained stable thereafter and between 20^{th} and 25th minute it equaled 9.16±2.13% ID/cc (P=0.3) (FIGS. 2A and D blue line). There was negligible signal observed in the contralateral hemisphere. ⁸⁹ZrBVDFO IA infusion followed by BBBO resulted in faster and significantly higher uptake of radioactivity in the ipsilateral hemisphere and it reached 23.58±4.58% ID/cc between 1st and 6th minute after infusion was completed, and signal remained stable thereafter and it was at 23.58±4.46% ID/cc (P=0.99) (FIGS. 2 B and D red line). Similarly to IA/BBBI group, no radioactivity accumulation in contralateral hemisphere was observed. In contrast, there was no preferential radioactivity uptake upon IV infusion of ⁸⁹ZrBVDFO in any hemisphere before and after BBBO and only background radioactivity was detected in the entire brain (before BBBO 2.91 and after 2.91% ID/cc, P=0.99), (FIGS. 2 C and D grey line). As expected, the gradual increase of radioactivity during infusions in the heart of mice belonging to all three groups was detected, with subsequent signal stabilization (FIG. 2 E). FIG. 3 contains representative PET images with overlaid mouse brain template available in the PMOD 3.4 and associated bar graph illustrating difference in accumulation ⁸⁹ZrBVDFO in different brain regions 1 h post infusion. Significantly higher accumulation of ⁸⁹ZrBVDFO in the brain was observed in the BBBO/IA group compared to two other groups with the highest radioactivity uptake in right striatum (16.92±5.7% ID/cc), right hippocampus (15.64±3.15% ID/cc) and right amygdala (12.27±2.77% ID/cc). In IA/BBBI group the highest uptake of ⁸⁹ZrBVDFO in the right hippocampus reaching only 8.4±1.75% ID/cc, In contrast, negligible uptake of radioactivity in all brain regions was detected upon IV infusion of ⁸⁹ZrBVDFO, followed by BBBO.

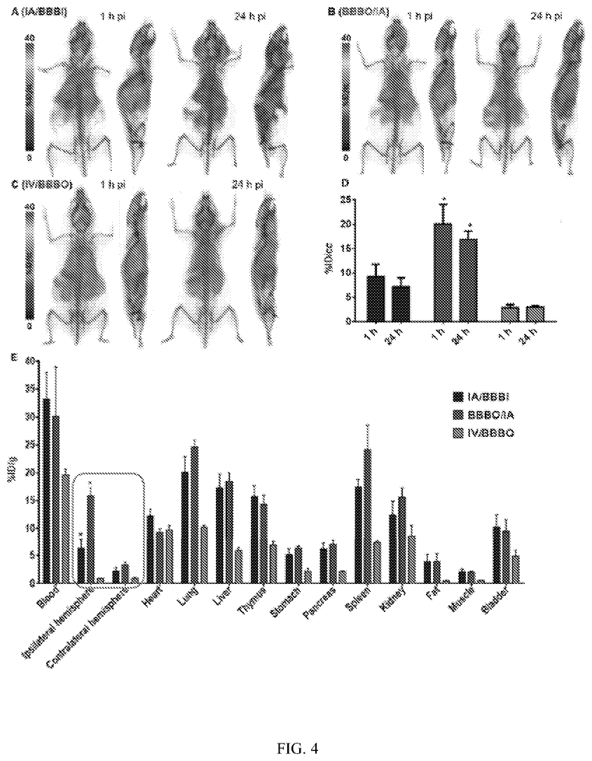

In agreement with dynamic scans, whole body PET-CT imaging recorded 1 and 24 h post infusion (FIGS. 4A, B and C) revealed the highest brain accumulation of ⁸⁹ZrBVDFO upon BBBO with mannitol, followed by its immediate IA infusion reaching 20.44±3.29% ID/cc and 16.91±1.67% ID/cc at 1 h and 24 h pi, respectively. IA infusion of ⁸⁹ZrBVDFO with BBBI resulted in accumulation of 9.25±2.54% ID/cc and 7.18±2.17% ID/cc in right hemisphere at 1 h and 24 h pi, respectively. BBBO with mannitol 10 min after IV infusion of ⁸⁹ZrBVDFO did not facilitate radioactivity uptake in the brain at 1 h and 24 h pi. Due to long circulation time of ⁸⁹ZrBVDFO, relatively high radioactivity background, (heart and lungs) was observed in all three groups. There was also accumulation of ⁸⁹ZrBVDFO around the neck 24 h post infusion, most likely due to surgical access for catheter placement triggering wound healing involving neovascularization.

### Ex Vivo Biodistribution

To validate PET-CT imaging results, ⁸⁹ZrBVDFO was further evaluated in ex vivo biodistribution analysis (FIG. 4E). As expected, we observed high accumulation of ⁸⁹ZrBVDFO in the ipsilateral hemisphere with % ID/g of 15.83±2.46 and only 2.29±0.82% ID/g in the contralateral hemisphere upon BBBO and IA infusion. IA infusion of 89ZrBVDFO with BBBI resulted in accumulation of 6.23±2.71% ID/g and 1.59±1.19% ID/g in ipsilateral and contralateral hemisphere, respectively. Uptake of ⁸⁹ZrBVDFO in both hemispheres was below 1% ID/g in animals treated with IV/BBBO. In agreement with earlier studies, high radioactivity level was detected in blood, lungs, spleen, liver and thymus (23).

### DISCUSSION

We observed a linear increase in concentration of ⁸⁹ZrBVDFO in the brain during IA infusion even with intact BBB, which maintained until 24 h pi. That is radically different compared to iron oxide nanoparticles or small molecules such as salicylic acid derivatives, which immediately clear from cerebral circulation after IA infusion (24). The osmotic BBBO strongly enhanced the uptake of ⁸⁹ZrBVDFO only after IA infusion, while it did not facilitate uptake of the radiotracer infused intravenously. IV delivery of ⁸⁹ZrBVDFO did not result in any cerebral uptake in naïve mice regardless of BBB status, in agreement with a similar study in mice bearing an orthotopic model of diffuse intrinsic pontine glioma, where no accumulation of ⁸⁹ZrBVDFO neither in the brain nor tumors upon its intravenous administration was observed (25). IV delivery of ⁸⁹ZrBVDFO two weeks after irradiation revealed some uptake in five out of seven patients with diffuse intrinsic pontine glioma, but it was characterized by the high heterogeneity and it only loosely correlated with MR enhancement territories (26). Interestingly, there was no specific signal in the brain 1 h after infusion but subsequent increase in signal was observed over the next 144 hours. Observed uptake of ⁸⁹ZrBVDFO might be rather related to the radiation-induced vascular injury and subsequent VEGF expression than the tumor specific accumulation.

The superiority of IA delivery presented in our study is well aligned with the rapidly growing applications for endovascular neurointerventions such as thrombectomy for ischemic stroke (27). The recently described method for highly predictable and spatially precise targeting of stem cells (28) and territory of BBB opening (29) using real-time MRI guidance promotes wider applications of endovascular neurointerventions beyond the vascular diseases.

### REFERENCES FOR EXAMPLE 1

1. Shergalis A, Bankhead A, 3rd, Luesakul U, Muangsin N, Neamati N. Current Challenges and Opportunities in Treating Glioblastoma. Pharmacol Rev. 2018; 70:412-445.
2. Jahangiri A, Chin A T, Flanigan P M, Chen R, Bankiewicz K, Aghi M K. Convectionenhanced delivery in glioblastoma: a review of preclinical and clinical studies. J Neurosurg. 2017; 126:191-200.
3. Grossman R, Burger P, Soudry E, et al. MGMT inactivation and clinical response in newly diagnosed GBM patients treated with Gliadel. J Clin Neurosci. 2015; 22:1938-1942.
4. Morshed R A, Cheng Y, Auffinger B, Wegscheid M L, Lesniak M S. The potential of polymeric micelles in the context of glioblastoma therapy. Front Pharmacol. 2013; 4:157.
5. Hersh D S, Kim A J, Winkles J A, Eisenberg H M, Woodworth G F, Frenkel V. Emerging Applications of Therapeutic Ultrasound in Neuro-oncology: Moving Beyond Tumor Ablation. Neurosurgery. 2016; 79:643-654.
6. Burkhardt J K, Riina H A, Shin B J, Moliterno J A, Hofstetter C P, Boockvar J A. Intra-arterial chemotherapy for malignant gliomas: a critical analysis. Interv Neuroradiol. 2011; 17:286-295.
7. Owens G. Arterial perfusion of the isolated canine brain. Am J Physiol. 1959; 197:475-477.
8. Hatiboglu I, Owens G. Results of intermittent, prolonged infusion of nitrogen mustard into the carotid artery in twelve patients with cerebral gliomas. Surg Forum. 1961; 12:396-398.
9. Neuwelt E A, Frenkel E P, Diehl J T, et al. Osmotic blood-brain barrier disruption: a new means of increasing chemotherapeutic agent delivery. Trans Am Neurol Assoc. 1979; 104:256-260.
10. Shapiro W R, Green S B, Burger P C, et al. A Randomized Comparison of Intraarterial Versus Intravenous Bcnu, with or without Intravenous 5-Fluorouracil, for Newly Diagnosed Patients with Malignant Glioma. Journal of Neurosurgery. 1992; 76:772-781.
11. Riina H A, Fraser J F, Fralin S, Knopman J, Scheff R J, Boockvar J A. Superselective intraarterial cerebral infusion of bevacizumab: a revival of interventional neuro-oncology for malignant glioma. J Exp Ther Oncol. 2009; 8:145-150.
12. Joshi S, Ellis J A, Ornstein E, Bruce J N. Intraarterial drug delivery for glioblastoma mutiforme: Will the phoenix rise again? J Neurooncol. 2015; 124:333-343.
13. Burkhardt J K, Santillan A, Hofstetter C P, et al. Intra-arterial bevacizumab with blood brain barrier disruption in a glioblastoma xenograft model. J Exp Ther Oncol. 2012; 10:31-37.
14. Chakraborty S, Filippi C G, Burkhardt J K, et al. Durability of single dose intra-arterial bevacizumab after blood/brain barrier disruption for recurrent glioblastoma. J Exp Ther Oncol. 2016; 11:261-267.
15. Mammatas L H, Verheul H M, Hendrikse N H, Yaqub M, Lammertsma A A, Menke-van der Houven van Oordt C W. Molecular imaging of targeted therapies with positron emission tomography: the visualization of personalized cancer care. Cell Oncol (Dordr). 2015; 38:49-64.
16. Vosjan M J, Perk L R, Visser G W, et al. Conjugation and radiolabeling of monoclonal antibodies with zirconium-89 for PET imaging using the bifunctional chelate pisothiocyanatobenzyl-desferrioxamine. Nat Protoc. 2010; 5:739-743.
17. Gaykema S B, Brouwers A H, Lub-de Hooge M N, et al. 89Zr-bevacizumab PET imaging in primary breast cancer. J Nucl Med. 2013; 54:1014-1018.
18. van Es S C, Brouwers A H, Mahesh S V K, et al. (89)Zr-Bevacizumab PET: Potential Early Indicator of Everolimus Efficacy in Patients with Metastatic Renal Cell Carcinoma. J Nucl Med. 2017; 58:905-910.
19. Bahce I, Huisman M C, Verwer E E, et al. Pilot study of Zr-89-bevacizumab positron emission tomography in patients with advanced non-small cell lung cancer. Ejnmmi Research. 2014; 4.
20. Jansen M, van Zanten S V, van Vuurden D, et al. Molecular Drug Imaging: (89)Zr-Bevacizumab Pet in Children with Diffuse Intrinsic Pontine Glioma. Neuro-Oncology. 2016; 18:57-57.
21. Jansen M H A, Lagerweij T, Sewing A C P, et al. Bevacizumab Targeting Diffuse Intrinsic Pontine Glioma: Results of Zr-89-Bevacizumab PET Imaging in Brain Tumor Models. Molecular Cancer Therapeutics. 2016; 15:2166-2174.
22. Jablonska A, Shea D J, Cao S, et al. Overexpression of VLA-4 in glial-restricted precursors enhances their endothelial docking and induces diapedesis in a mouse stroke model. J Cereb Blood Flow Metab. 2018; 38:835-846.
23. Nagengast W B, de Vries E G, Hospers G A, et al. In vivo VEGF imaging with radiolabeled bevacizumab in a human ovarian tumor xenograft. J Nucl Med. 2007; 48:1313-1319.
24. Song X, Walczak P, He X, et al. Salicylic acid analogues as chemical exchange saturation transfer MRI contrast agents for the assessment of brain perfusion territory and blood-brain barrier opening after intra-arterial infusion. J Cereb Blood Flow Metab. 2016; 36:1186-1194.
25. Jansen M H, Lagerweij T, Sewing A C, et al. Bevacizumab Targeting Diffuse Intrinsic Pontine Glioma: Results of 89Zr-Bevacizumab PET Imaging in Brain Tumor Models. Mol Cancer Ther. 2016; 15:2166-2174.
26. Jansen M H, Veldhuijzen van Zanten S E M, van Vuurden D G, et al. Molecular Drug Imaging: (89)Zr-Bevacizumab PET in Children with Diffuse Intrinsic Pontine Glioma. J Nucl Med. 2017; 58:711-716.
27. Jovin T G, Chamorro A, Cobo E, et al. Thrombectomy within 8 hours after symptom onset in ischemic stroke. N Engl J Med 2015; 372:2296-2306.
28. Walczak P, Wojtkiewicz J, Nowakowski A, et al. Real-time MRI for precise and predictable intra-arterial stem cell delivery to the central nervous system. J Cereb Blood Flow Metab. 2017; 37:2346-2358.
29. Janowski M, Walczak P, Pearl M S. Predicting and optimizing the territory of blood-brain barrier opening by superselective intra-arterial cerebral infusion under dynamic susceptibility contrast MRI guidance. J Cereb Blood Flow Metab. 2016; 36:569-575.
30. Liu H, Jablonska A, Li Y, et al. Label-free CEST MRI Detection of Citicoline-Liposome Drug Delivery in Ischemic Stroke. Theranostics. 2016; 6:1588-1600.

### Example 2

### Materials

All chemicals were purchased from Sigma-Aldrich (Milwaukee, Wis.) or Fisher Scientific (Tewksbury, Mass.) unless otherwise specified. Ethylenediamine core amineterminated generation-4 poly(amidoamine dendrimer) [G4(NH₂)₆₄] was acquired from Dendritech (Midland, Mich.). ⁸⁹Zr(C₂O₄)₂ (t_{1/2}=78.4 h) and 1-(4-isothiocyanatophenyl)-3-[6,17-dihydroxy-7,10,18,21-tetraoxo-27-(N-acetylhydroxylamino)-6,11,17, 22-tetraazaheptaeicosine] thiourea (p-SCN-Bn-DFO, Cat. #B-705) were obtained from Washington University (St. Louis, Mo.) and Macrocyclics (Plano, Tex.), respectively. All reagents and solvents were used as received without further purification.

### Nanobody

Gelsolin nanobody 11, cloned in the pHEN6c vector, was purified from WK6 cells as described previously [18]. Briefly, competent WK6 cells were transformed with the plasmid and grown at 37° C. in TB medium with 100 μg/mL ampicillin until the OD600 reached 0.60-0.80. Then temperature was set to 20° C. and nanobody expression was induced by the addition of 0.5 mM IPTG. After overnight induction, bacterial cultures were pelleted by centrifugation at 11,000×g for 20 min at 4° C. Cells were resuspended in a small volume of phosphate buffered saline (PBS) and 0.2 mg/mL lysozyme was added. Lysis proceeded during 30 min rotation at room temperature. This suspension was then sonicated (Vibracell, Sonics and Materials, Newtown, USA) and centrifuged again (˜29,000×g) for 30 min at 4° C. to obtain the bacterial protein lysate. The His6-tagged nanobody was purified by Immobilized Metal ion Affinity Chromatography (IMAC) on a Ni2+ column and eluted with 500 mM imidazole. Finally, nanobody 11 was purified to homogeneity by gel filtration chromatography on a Superdex 200 HR 10/30 column (GE Healthcare, Diegem, Belgium), equilibrated in 20 mM Tris.HCl pH 7.5, 150 mM NaCl, 1 mM DTT.

### Synthesis of NB(DFO)₂

For conjugation of DFO with nanobody storage buffer was replaced with saline using ultrafiltration with Millipore Amicon Ultra Centrifugal Filters 3,000 Da molecular weight cut-off (MWCO, Millipore Sigma, cat #: UFC80030) and pH was adjusted to 9 with a small amount of 2 M Na₂CO₃ solution. Then five-fold molar equivalent of SCN-Bn-DFO dissolved in DMSO was added and conjugation was carried out for 30 min at 37° C. in a thermomixer at 550 r.p.m. Resulting NB-DFO conjugate was purified as described above, reconstituted in saline at 10 mg/mL and 0.1 mL aliquots were kept at −20° C. until further use.

Synthesis of G4(DFO)₃(Bdiol)₁₁₀ Dendrimer

Preparation of G4(DFO)₃(Bdiol)₁₁₀ involved a one pot two-step synthesis as presented in Scheme 2. G4(NH₂)₆₄ dendrimer (0.030 g, 2.11×10−6 mol) was dissolved in 3 mL deionized water resulting in pH=9.2 and 5 mol equivalent of SCN-Bn-DFO (0.008 g, 1.05×10-5 mol) reconstituted in 0.2 mL of DMSO was added. The reaction proceeded for 30 min at 37° C. in a thermomixer at 550 r.p.m. and a small amount of reaction mixture was subjected to MALDI-TOF mass spectrometry to confirm conjugation of DFO with dendrimer. Next, 0.2 mL (2.99×10−3 mol) of glycidol was added and reaction was carried for additional overnight to cap remaining primary amines with butane-1,2-diol (Bdiol). Resulting G4(DFO)₃(Bdiol)₁₁₀ dendrimer was purified using deionized water and ultrafiltration with Millipore Amicon Ultra Centrifugal Filters 10,000 Da MWCO, lyophilized, yielding 0.035 g of the conjugate, which was stored −20° C. until further use. Matrix-Assisted Laser Desorption Ionization-Time-of-Flight (MALDI-TOF)

To determine average number of DFO molecules conjugated with nanobody and dendrimer and assess its capping efficiency with butane-1,2-dio, 1 MALDI-TOF spectra were recorded on a Voyager DE-STR spectrophotometer, using 2,5-dihydroxybenzoic acid (DHB) as a matrix, which was dissolved in 50% MeOH and 0.1% TFA aqueous solution at concentration of 20 mg/mL. NB and NB(DFO)₂ samples were desalted using Zeba™ spin columns 7K MWCO (cat. #89882, Thermo Fisher Scientific). Samples of G4(NH₂)₆₄, G4(NH₂)₆₁, (DFO)₃ and G4(DFO)₃(Bdiol)₁₁₀ dendrimers were prepared in deionized water. 10 μL of samples were mixed with 10 μL of matrix and 1 μL of resulting mixture was placed on the target plate (in triplicate) and evaporated. Number of shots and laser power was adjusted according to spectrum quality.

### Dynamic Light Scattering and Zeta Potential Analysis

Dynamic light scattering and zeta potential analyses were performed using a Malvern Zetasizer Nano ZEN3600. G4(DFO)₃(Bdiol)₁₁₀ dendrimer was prepared at a concentration of 4 mg/mL in PBS (c=0.1 M, pH 7.4). DLS measurements were performed at a 90° scattering angle at 25° C.

Radiolabeling of NB(DFO)₂ and G4(DFO)₃(Bdiol)₁₁₀

Radiolabeling of NB(DFO)₂ and G4(DFO)₃(Bdiol)₁₁₀ with ⁸⁹Zr was performed using reported procedure [19]. ⁸⁹ZrNB(DFO)₂ was fabricated with ˜99% radiochemical purity and 129.5±10 MBq/mg specific activity. ⁸⁹ZrG4(DFO)₃(Bdiol)¹¹⁰ was prepared with ˜99% radiochemical purity and 120±8 MBq/mg specific activity. For further studies ⁸⁹ZrNB(DFO)₂ and ⁸⁹ZrG4(DFO)₃(Bdiol)¹¹⁰ were diluted with sterile saline. PET-CT imaging of IA and IV delivery of ⁸⁹ZrNB(DFO)₂ and ⁸⁹ZrG₄(DFO)₃(Bdiol)₁₁₀ with or without OBBBO.

PET-CT studies were performed as we have recently described [6]. Briefly, under general anesthesia catheter was placed in the internal carotid artery (ICA) and mice were transferred to the PET-CT scanner. BBB opening was performed with 25% mannitol infused for 1 min at a speed of 0.15 mL/min. ˜8.5 MBq (˜230 μCi) ⁸⁹ZrNB(DFO)₂ or ⁸⁹ZrG₄(DFO)₃(Bdiol)₁₁₀ reconstituted in 1 mL of saline was infused IA or IV over 5 min at 0.15 mL/min flow rate. Thus, there were three experimental groups: 1) IA infusion with BBB intact (IA/BBBI), 2) OBBBO followed by IA infusion (OBBBO/IA), and 3) Intravenous infusion followed by OBBBO (IV/OBBBO). Accumulation of ⁸⁹ZrNB(DFO)₂ or ⁸⁹ZrG₄(DFO)₃(Bdiol)₁₁₀ in the brain was initially evaluated with dynamic 30 min long PET scans divided into 30 second frames and followed by whole body PET/CT imaging acquired around 1 h and 24 h post-infusion (pi), in two bed positions and 7 min per bed on an ARGUS small-animal PET/CT scanner (Sedecal, Madrid, Spain). A CT scan (512 projections) was performed before whole body PET imaging at 1 h (mice remained in the scanner after dynamic scan was completed) and 24 h pi, to enable co-registration. PET data were reconstructed using the two-dimensional ordered subsets-expectation maximization algorithm (2D-OSEM) and corrected for dead time and radioactive decay. Presented whole body images were generated using Amira® (FEI, Hillsboro, Oreg.) and dynamic scans (brain and heart radioactivity accumulation) and radioactivity distribution in different brain regions were analyzed with PMOD 4.3 (PMOD Technologies LLC, Zurich, Switzerland). The peak concentration of radioactivity over 5 min around the end of IA infusion of 89ZrNB(DFO)₂ and ⁸⁹ZrG₄(DFO)₃(Bdiol)₁₁₀ was extracted and compared with the last 5 min of the dynamic scans to calculate the rate of early clearance of administrated radiotracers from the brain. Then the radioactivity detected in the CNS at 1 h and 24 h after infusion was used to assess their later brain clearance. The effect of OBBBO on nanobody or dendrimer brain accumulation following their IV infusion was evaluated by comparing level of radioactivity 5 min before and 5 min after mannitol administration.

Ex Vivo Biodistribution of ⁸⁹ZrNB(DFO)₂ and ⁸⁹ZrG₄(DFO)₃(Bdiol)₁₁₀

Upon completion of PET-CT at 24 h pi of ⁸⁹ZrNB(DFO)₂ or ⁸⁹ZrG₄(DFO)₃(Bdiol)₁₁₀ mice were sacrificed, blood, brain (divided into right and left hemispheres) and selected organs were harvested and weighed. The radioactivity in collected samples was measured on a PerkinElmer—2480 Automatic Gamma Counter (Waltham, Mass.) four days after sample collection to avoid detector saturation due to high radioactivity accumulation in brain and kidneys. To calculate the percent of injected dose per gram of tissue (% ID/g), triplicate radioactive standards (0.01% of the injected dose) were counted along with tissue samples. Biodistribution data shown is mean±the standard deviation (SD).

### Statistical Analysis

PROC MIXED (SAS 9.4) was used for statistical analysis, with the lowest means square (LMS) test for comparison between groups. The statements “repeated” and “random” were used for repeated measures and to express random effects, respectively.

### Results

Synthesis of ⁸⁹ZrNB(DFO)₂

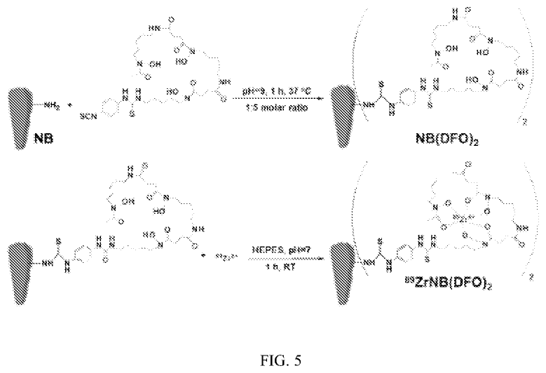

Preparation of ⁸⁹ZrNB(DFO)₂ involved conjugation of on average two DFO molecules as measured by MALDI-TOF spectrometry and subsequent radiolabeling with ⁸⁹Zr (FIG. 5).

### CNS Uptake of ⁸⁹ZrNB(DFO)2 and its Biodistribution

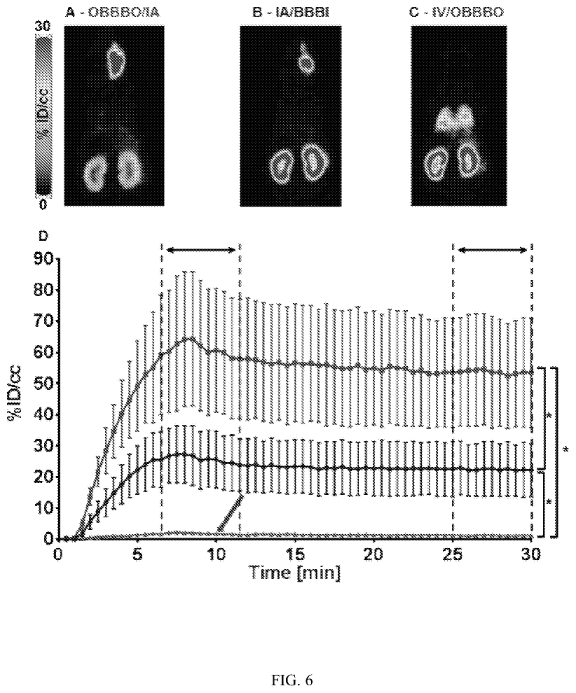

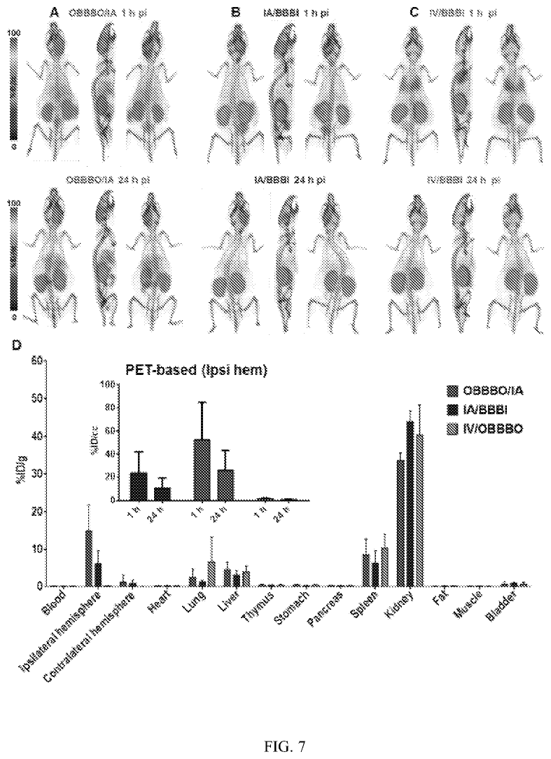

Nearly linear uptake of radioactivity in the ipsilateral hemisphere was observed during IA infusions of 89ZrNB(DFO)2 regardless of the BBB status, with no accumulation in the contralateral hemisphere (FIG. 6). The IA/BBBI infusion resulted in ⁸⁹ZrNB(DFO)₂ accumulation in the ipsilateral hemisphere with a peak concentration of 25.79±15.79% ID/cc and OBBBO further enhanced its uptake to 60.66±35.41% ID/cc (P<0.05). Only background radioactivity was observed in the CNS after IV infusion (1.93±0.31% ID/cc), which actually decreased after OBBBO to (1.59±0.26% ID/cc, P<0.05). There was very slow early clearance of radioactivity from the ipsilateral hemisphere observed over a period of 30 min, which was not-significant for IA/BBBI (22.46±15.05, P=NS), but it was statistically different for OBBBO/IA infusion (53.66±30.73, P<0.05). The background radioactivity after IV/OBBBO was not changed at the end of infusion (1.29±0.25, P=NS). The whole-body PET-CT imaging performed 1 h after infusion revealed a similar pattern of radioactivity uptake in the brain as at the end of the dynamic PET scan, which then decreased nearly by half 24 h after infusion (P<0.05). In all evaluated cohorts high uptake of radioactivity was also observed in kidney, indicating fast renal clearance. High accumulation of ⁸⁹ZrNB(DFO)₂ in the ipsilateral hemisphere upon OBBBO/IA infusion resulted in its statistically relevant lower concentration in kidneys at 1 h after infusion (26.72±4.19) in comparison to IA/BBBI (43.36±3.83) and IV/OBBBO (39.61±7.51% ID/cc). The clearance of ⁸⁹ZrNB(DFO)₂ from brain over 24 h resulted in increase of radioactivity in kidneys to 35.38±5.11% ID/cc in OBBBO/IA group (P<0.05), while no difference was observed for the remaining experimental groups (41.84±5.47 and 40.34%±7.91% ID/cc for IA/BBBI and OBBBO/IV, respectively). For IV/OBBBO infusion 12.48±2.32% ID/cc of ⁸⁹ZrNB(DFO)₂ could also be detected in the lungs 1 h after infusion. In agreement with PET-CT imaging, post mortem biodistribution analysis revealed significantly higher accumulation of ⁸⁹ZrNB(DFO)₂ in the ipsilateral hemisphere in OBBBO/IA (17.8±5.99% ID/g) compared to IA/BBBI (6.15±3.53% ID/g) and IV/OBBBO (0.09±0.03% ID/g) infusions with negligible radioactivity uptake in the contralateral hemispheres in all mice 24 h after infusion. Among peripheral organs the highest accumulation of ⁸⁹ZrNB(DFO)₂ was detected in kidneys followed by the spleen, liver and lungs.

Synthesis of ⁸⁹ZrG₄(DFO)₃(Bdiol)₁₁₀

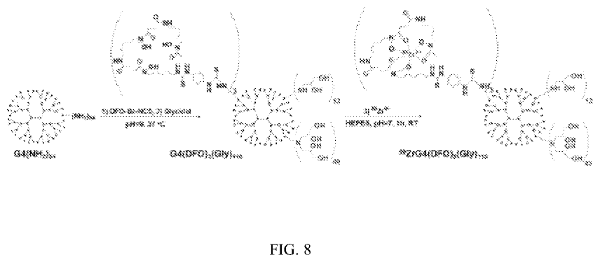

G4(NH₂)₆₄ was conjugated with average three molecules of DFO (FIG. 8) and remaining primary amines were substituted with 110 butane-1,2-diol moieties, assessed by increase of the molecular weight observed in MALDI-TOF spectrometry (FIG. S2A). A one-pot synthesis yielded nanoparticles with narrow size distribution around 5 nm (FIG. S2B) and neutral net-surface charge, indicated by zeta potential of −1.8 mV. Resulting G₄(DFO)₃(Bdiol)₁₁₀ dendrimer was subsequently radiolabeled with 89Zr and used for further studies.

CNS Uptake of ⁸⁹ZrG₄(DFO)₃(Bdiol)₁₁₀ and its Biodistribution

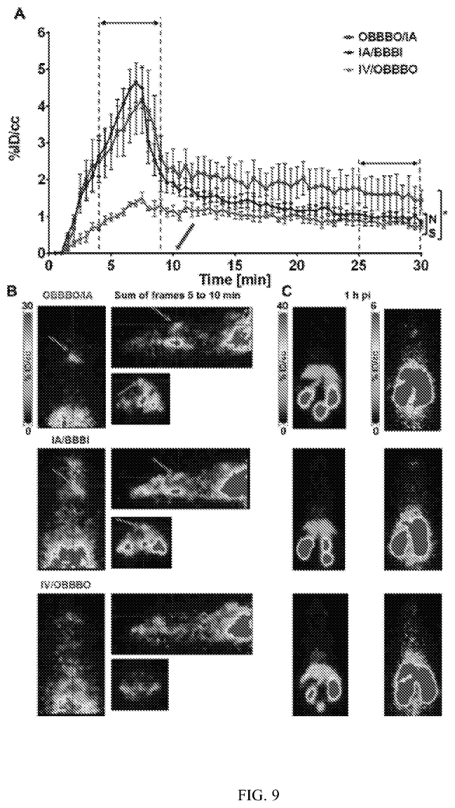

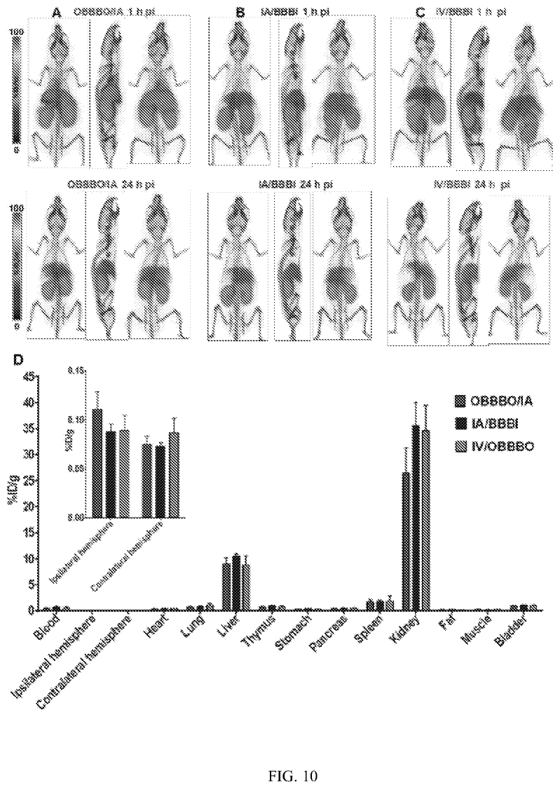

There was no difference in the peak concentration of ⁸⁹ZrG₄(DFO)₃(Bdiol)₁₁₀ in the ipsilateral hemisphere for IA/BBBI (3.29±1.31% ID/cc) and OBBBO/IA (3.20±1.47% ID/cc) infusions (P=NS) as indicated by the time activity curves and PET images obtained by summing frames collected between 5 and 10 min of dynamic scans (FIG. 9). IV/OBBBO infusion resulted in a background radioactivity uptake of 1.22±0.29% ID/cc in the CNS, with decrease of radioactivity after OBBBO to 1.1±0.25 (P<0.05). The fast and statistically significant clearance of ⁸⁹ZrG₄(DFO)₃(Bdiol)₁₁₀ from the brain was observed regardless of BBB status and it reached 1.68±0.8, 1.05±0.22, 0.83±0.018% ID/cc for OBBBO/IA, IA/BBBI and OBBBO/IV, at the end of the dynamic PET scan, respectively. However, the clearance after OBBBO/IA was somewhat slower compared to IA/BBBI (P<0.05). IA/BBBI actually dropped to the same low level as IV/OBBBO (P=NS) at the end of dynamic scans. However, the whole-body PET-CT imaging performed 1 h after infusion showed only background radioactivity in the brain regardless of the route of ⁸⁹ZrG₄(DFO)₃(Bdiol)₁₁₀ delivery with no statistically significant differences among groups (FIG. 10). Significant amounts of radioactivity could be detected in kidneys and bladder, followed by liver at 1 h after infusion, indicating fast renal clearance with minor hepatic involvement. At 24 h after infusion no radioactivity in the brain of all evaluated mice was observed. In agreement with PET-CT imaging post mortem biodistribution demonstrated negligible accumulation of ⁸⁹ZrG₄(DFO)₃(Bdiol)₁₁₀ in both hemispheres (P=NS) and exclusive presence of radioactivity in kidneys and liver for all assessed delivery routes (FIG. 10). 24 h after infusion radioactivity in the ipsilateral hemisphere and bladder was below PET quantification limit.

### Discussion

We have shown that the IA route was more effective in delivering nanobodies to the brain than systemic administration. Preceding OBBBO potentiated brain accumulation of the nanobodies by ˜2.5-fold. Brain uptake of ⁸⁹ZrNB(DFO)₂ reached 60.66±35.41% ID/cc, which is higher compared to brain accumulation of 23.58±4.58% ID/cc for 89Zr radiolabeled-bevacizumab (⁸⁹ZrBVDFO) observed in our previous study [6]. While half of the ⁸⁹ZrNB(DFO)₂ was cleared from the brain over 24 h, clearance of ⁸⁹ZrBVDFO was slower. In both studies bevacizumab and nanobody did not have specific targets in mouse brains. In contrast, brain retention of generation-4 hydroxy terminated PAMAM dendrimer was marginal. The peak concentration of ⁸⁹ZrG₄(DFO)₃(Bdiol)₁₁₀ in the brain was only around 3% ID/cc after IA delivery regardless BBB status and decreased to background levels within 1 h. Intravenous infusion of ⁸⁹ZrNB(DFO)₂ and ⁸⁹ZrG₄(DFO)₃(Bdiol)₁₁₀ resulted in only background radioactivity regardless of BBB status. Our results are in agreement with previous reports showing negligible penetration of PAMAM dendrimers through intact BBB upon IV administration, regardless of their size and terminal functionalities, including hydroxy, carboxyl and polyethylene glycol groups [20-22]. Kannan et al. demonstrated uniform accumulation of Cy5 fluorescently labeled generation-4 hydroxy terminated PAMAM dendrimer in a rodent model of gliosarcoma, as well as its specific uptake by tumor-associated macrophages after systemic delivery [16]. Although microscopic imaging was convincing, the peak concentration of dendrimer in tumor reached only 0.023% ID/g at 8 h after injection and decreased to 0.0067% ID/g 40 h later, as measured by fluorescence spectroscopy of extracted tissue [16]. Similarly, very low brain uptake of ˜0.07% ID/g in neonatal rabbits with cerebral palsy and 0.003% ID/g healthy control pups for the same dendrimer at 24 h after injection was also reported [23]. Both studies, in agreement with our results, demonstrated marginal BBB permeability and brain retention of generation-4 hydroxy terminated PAMAM dendrimer even with a compromised blood brain barrier, brain tumor or activated microglia present in cerebral palsy model. Interestingly, PET imaging of generation-4 hydroxy terminated dendrimer-radiolabeled with copper-64 in newborn rabbits with cerebral palsy indicated brain accumulation of radioactivity around 2.5% ID/cc 24 h after injection [14]. However, copper-64 undergoes trans-chelation in vivo, in particular in the absence of a strong Cu(II) chelator forming thermodynamically stable complexes [24].

Our ⁸⁹ZrNB(DFO)₂ and ⁸⁹ZrG₄(DFO)₃(Bdiol)₁₁₀ were not targeted to specific molecular species within brain. Also, no disease model was induced, enabling testing as a baseline therapeutic delivery platform for CNS drug delivery. In this context, the nanobodies seems attractive for IA infusion, while a lot of caution should be taken regarding utility of PAMAM dendrimers as drug delivery vehicles for brain diseases, especially when they are administered systemically. Therefore in case of PAMAM dendrimers the challenge for appropriate surface modification to achieve appreciate brain uptake and retention remains open. While here we tested generation-4 hydroxy terminated PAMAM dendrimers constructed by capping the primary amines with butane-1,2-diol, the same dendrimers with different surface modifications can potentially exhibit higher brain retention and our study may serve as a benchmark for quantitative performance of dendrimer-based diagnostics and therapeutics in the CNS diseases. In contrast, IA route is very effective in delivery of nanobodies and their relatively fast clearance comparing to antibody could potentially be mitigated by applying nanobodies aimed for specific brain target. While, IV administration is highly ineffective for delivery of nanobodies to the brain, it was recently reported that intranasal route might be an alternative [25]. However, no quantitative assessment of intranasal brain delivery of nanobodies has been reported yet. There is a progress in design of nanobodies against brain disorders [26], and our IA infusion might be a right approach to use them effectively in the clinic. Especially, after the anti-tumoral activity of neutralizing antibodies was shown in a mouse model of melanoma, the potentially neutralizing nanobodies could also be created against brain targets [27].

Limitations: We have observed relatively high variability in brain uptake of nanobodies after IA delivery. We performed four rounds of experiments, in three groups of animals (IA/BBBI, OBBBO/IA and OBBBO/IV) and while we observed high reproducibility within rounds with relatively constant ratio of brain uptake OBBBO/IA versus IA/BBBI (ca. 2.5×), relatively high variability between rounds was observed. Interestingly, in one animal we have observed the brain uptake at the level of nearly 100% ID/cc, which actually shows a high promise of IA route and possibility for further improvement of nanobody delivery to the brain. There might be various sources of variability including kinetics of cerebral blood flow or volume of the brain perfused from the IA catheter. It has been recently shown that real-time MRI can increase reproducibility of OBBBO [28], thus studies like ours would benefit from PET/MR systems, in which infusion parameters could be adjusted based on feedback from real-time MRI and quantitative assessment of brain uptake of infused molecules based on PET imaging. In clinical setting the real-time monitoring of IA delivery of nanobodies to the brain using PET, until the required quantity is achieved, might be an ultimate solution for precise dosing. In our study we have not measured the affinity of radiolabeled nanobody, as we have not used it to bind specific target, but it was previously shown that nanobodies can be radiolabeled without losing their efficacy providing a proof-of-concept for a viability of our approach [29, 30]. Also, we have not studied the reasons for such different penetration of BBB by two similar size molecules: nanobodies and dendrimers, however such experiments are warranted and should be performed in the future to better understand rules governing an advantage of IA delivery of macromolecules.

### Conclusions

We have shown that brain delivery of nanobodies and generation-4 hydroxy terminated PAMAM dendrimers upon IV administration is negligible regardless of BBB status. The IA route substantially increases brain uptake of nanobodies, which is further potentiated by OBBBO. However, half of nanobodies are cleared from the brain within 24 h. Designing nanobodies against specific brain targets could ameliorate this deficiency. In contrast, the IA route marginally improved brain delivery of dendrimers, which quickly cleared from CNS. Appropriate surface modification of PAMAM dendrimers may improve their brain uptake and retention.

### REFERENCES FOR EXAMPLE 2

1. Woodworth G F, Dunn G P, Nance E A, Hanes J, Brem H. Emerging insights into barriers to effective brain tumor therapeutics. Frontiers in oncology. 2014; 4:126. doi:10.3389/fonc.2014.00126.
2. Mayhan W G, Heistad D D. Permeability of blood-brain barrier to various sized molecules. The American journal of physiology. 1985; 248:H712-8. doi:10.1152/ajpheart.1985.248.5.H712.
3. On N H, Miller D W. Transporter-based delivery of anticancer drugs to the brain: improving brain penetration by minimizing drug efflux at the blood-brain barrier. Current pharmaceutical design. 2014; 20:1499-509.
4. Oldendorf W H. Lipid solubility and drug penetration of the blood brain barrier. Proceedings of the Society for Experimental Biology and Medicine Society for Experimental Biology and Medicine. 1974; 147:813-5.
5. Boockvar J A, Tsiouris A J, Hofstetter C P, Kovanlikaya I, Fralin S, Kesavabhotla K, et al. Safety and maximum tolerated dose of superselective intraarterial cerebral infusion of bevacizumab after osmotic blood-brain barrier disruption for recurrent malignant glioma. Clinical article. Journal of neurosurgery. 2011; 114:624-32. doi:10.3171/2010.9.JNS101223.
6. Lesniak W G, Chu C, Jablonska A, Du Y, Pomper M G, Walczak P, et al. PET imaging of intra-arterial (89)Zr bevacizumab in mice with and without osmotic opening of the blood-brain barrier: distinct advantage of intra-arterial delivery. Journal of nuclear medicine: official publication, Society of Nuclear Medicine. 2018. doi:10.2967/jnumed.118.218792.
7. Banks W A. Characteristics of compounds that cross the blood-brain barrier. BMC neurology. 2009; 9 Suppl 1:S3. doi:10.1186/1471-2377-9-S1-S3.
8. Janowski M, Walczak P, Pearl M S. Predicting and optimizing the territory of bloodbrain barrier opening by superselective intra-arterial cerebral infusion under dynamic susceptibility contrast MRI guidance. Journal of cerebral blood flow and metabolism: official journal of the International Society of Cerebral Blood Flow and Metabolism. 2016; 36:569-75. doi:10.1177/0271678X15615875.
9. Walczak P, Wojtkiewicz J, Nowakowski A, Habich A, Holak P, Xu J, et al. Realtime MRI for precise and predictable intra-arterial stem cell delivery to the central nervous system. Journal of cerebral blood flow and metabolism: official journal of the International Society of Cerebral Blood Flow and Metabolism. 2017; 37:2346-58. doi:10.1177/0271678X16665853.
10. Ingram J R, Schmidt F I, Ploegh H L. Exploiting Nanobodies' Singular Traits. Annual review of immunology. 2018; 36:695-715. doi:10.1146/annurev-immunol-042617-053327.
11. Bannas P, Lenz A, Kunick V, Well L, Fumey W, Rissiek B, et al. Molecular imaging of tumors with nanobodies and antibodies: Timing and dosage are crucial factors for improved in vivo detection. Contrast media & molecular imaging. 2015; 10:367-78. doi:10.1002/cmmi.1637.
12. Kannan R M, Nance E, Kannan S, Tomalia D A. Emerging concepts in dendrimerbased nanomedicine: from design principles to clinical applications. Journal of internal medicine. 2014; 276:579-617. doi:10.1111/joim.12280.
13. Chauhan A S. Dendrimers for Drug Delivery. Molecules. 2018; 23. doi:10.3390/molecules23040938.
14. Kannan S, Dai H, Navath R S, Balakrishnan B, Jyoti A, Janisse J, et al. Dendrimerbased postnatal therapy for neuroinflammation and cerebral palsy in a rabbit model. Science translational medicine. 2012; 4:130ra46. doi:10.1126/scitranslmed.3003162.
15. Balakrishnan B, Nance E, Johnston M V, Kannan R, Kannan S. Nanomedicine in cerebral palsy. International journal of nanomedicine. 2013; 8:4183-95. doi:10.2147/IJN.535979.
16. Zhang F, Mastorakos P, Mishra M K, Mangraviti A, Hwang L, Zhou J, et al. Uniform brain tumor distribution and tumor associated macrophage targeting of systemically administered dendrimers. Biomaterials. 2015; 52:507-16. doi:10.1016/j.biomaterials.2015.02.053.
17. Qiu J, Kong L, Cao X, Li A, Wei P, Wang L, et al. Enhanced Delivery of Therapeutic siRNA into Glioblastoma Cells Using Dendrimer-Entrapped Gold Nanoparticles Conjugated with beta-Cyclodextrin. Nanomaterials. 2018; 8. doi:10.3390/nano8030131.
18. Van den Abbeele A, De Clercq S, De Ganck A, De Corte V, Van Loo B, Soror S H, et al. A llama-derived gelsolin single-domain antibody blocks gelsolin-G-actin interaction. Cell Mol Life Sci. 2010; 67:1519-35. doi:10.1007/s00018-010-0266-1.
19. Vosjan M J, Perk L R, Visser G W, Budde M, Jurek P, Kiefer G E, et al. Conjugation and radiolabeling of monoclonal antibodies with zirconium-89 for PET imaging using the bifunctional chelate p-isothiocyanatobenzyl-desferrioxamine. Nature protocols. 2010; 5:739-43. doi:10.1038/nprot.2010.13.
20. Laznickova A, Biricova V, Laznicek M, Hermann P. Mono(pyridine-N-oxide) DOTA analog and its G1/G4-PAMAM dendrimer conjugates labeled with 177Lu: radiolabeling and biodistribution studies. Applied radiation and isotopes: including data, instrumentation and methods for use in agriculture, industry and medicine. 2014; 84:70-7. doi:10.1016/j.apradiso.2013.10.021.
21. Sadekar S, Ray A, Janat-Amsbury M, Peterson C M, Ghandehari H. Comparative biodistribution of PAMAM dendrimers and HPMA copolymers in ovarian-tumor-bearing mice. Biomacromolecules. 2011; 12:88-96. doi:10.1021/bm101046d.
22. Zhang Y, Sun Y, Xu X, Zhang X, Zhu H, Huang L, et al. Synthesis, biodistribution, and microsingle photon emission computed tomography (SPECT) imaging study of technetium-99m labeled PEGylated dendrimer poly(amidoamine) (PAMAM)-folic acid conjugates. Journal of medicinal chemistry. 2010; 53:3262-72. doi:10.1021/jm901910j.
23. Lesniak W G, Mishra M K, Jyoti A, Balakrishnan B, Zhang F, Nance E, et al. Biodistribution of fluorescently labeled PAMAM dendrimers in neonatal rabbits: effect of neuroinflammation. Molecular pharmaceutics. 2013; 10:4560-71. doi:10.1021/mp400371r.
24. Boswell C A, Sun X, Niu W, Weisman G R, Wong E H, Rheingold A L, et al. Comparative in vivo stability of copper-64-labeled cross-bridged and conventional tetraazamacrocyclic complexes. Journal of medicinal chemistry. 2004; 47:1465-74. doi:10.1021/jm030383m.
25. Gomes J R, Cabrito I, Soares H R, Costelha S, Teixeira A, Wittelsberger A, et al. Delivery of an anti-transthyretin Nanobody to the brain through intranasal administration reveals transthyretin expression and secretion by motor neurons. Journal of neurochemistry. 2018; 145:393-408. doi:10.1111/jnc.14332.
26. Samec N, Jovcevska I, Stojan J, Zottel A, Liovic M, Myers M P, et al. Glioblastomaspecific anti-TUFM nanobody for in-vitro immunoimaging and cancer stem cell targeting. Oncotarget. 2018; 9:17282-99. doi:10.18632/oncotarget.24629.
27. McMurphy T, Xiao R, Magee D, Slater A, Zabeau L, Tavernier J, et al. The antitumor activity of a neutralizing nanobody targeting leptin receptor in a mouse model of melanoma. PloS one. 2014; 9:e89895. doi:10.1371/journal.pone.0089895.
28. Chu C, Liu G, Janowski M, Bulte J W M, Li S, Pearl M, et al. Real-Time MRI Guidance for Reproducible Hyperosmolar Opening of the Blood-Brain Barrier in Mice. Frontiers in Neurology. 2018; 9. doi:10.3389/fneur.2018.00921.
29. Vaidyanathan G, McDougald D, Choi J, Koumarianou E, Weitzel D, Osada T, et al. Preclinical Evaluation of 18F-Labeled Anti-HER2 Nanobody Conjugates for Imaging HER2 Receptor Expression by Immuno-PET. Journal of nuclear medicine: official publication, Society of Nuclear Medicine. 2016; 57:967-73. doi:10.2967/jnumed.115.171306.
30. Bala G, Blykers A, Xavier C, Descamps B, Broisat A, Ghezzi C, et al. Targeting of vascular cell adhesion molecule-1 by 18F-labelled nanobodies for PET/CT imaging of inflamed atherosclerotic plaques. European heart journal cardiovascular Imaging. 2016; 17:1001-8. doi:10.1093/ehjci/jev346.

### Example 3

Two-photon microscopy (2PM) is an intravital imaging technique that allows imaging of tissue up to about one millimeter in depth [17]. Using 2PM, it is achievable to reach sufficient temporal and spatial resolution in the cerebral cortex to track an agent's penetration across the BBB at the level of microvasculature. Due to limited depth penetration, 2PM studies are restricted to superficial structures such as cerebral cortex accessed with an implanted cranial window [18]. However, as we recently reported, OBBBO in mice with intracarotid mannitol infused at the hemodynamically safe rate of ˜0.15 ml/min is primarily routed to deep brain structures without perfusion through cerebral cortex [19]. Consequently, OBBBO does not consistently involve cerebral cortex. The phenomenon is likely due to specifics of blood supply and collateralization [20, 21]. As such, OBBBO has been out of reach for 2PM. The main motivation for this study was to develop an approach to enable OBBBO in the cerebral cortex. We hypothesized that the contralateral common carotid artery (CCA) compensates for the lost blood supply from catheterized ipsilateral CCA. Therefore, we explored with real-time MRI whether temporary occlusion of the contralateral CCA (cCCA) opens BBB in ipsilateral cortex, and subsequently validated capability of visualization of this process by intravital microscopy.

### Materials and Methods

### Animals and Endovascular Catheterization

All procedures were performed in accordance with guidelines for the care and use of laboratory animals and were approved by the Johns Hopkins Animal Care and Use Committee. Male SCID mice (n=26, 6-8 weeks old, 20-25 g, Jackson Laboratory) were used in this study. The surgical procedures for gaining arterial access were performed as described previously [19]. Briefly, anesthesia was induced with 5% isoflurane and maintained with 1.5-2% isoflurane during surgery. The CCA bifurcation was exposed using blunt dissection. The occipital artery branching off from the external carotid artery (ECA) was coagulated. The ECA and the pterygopalatine artery (PPA) were temporarily ligated with 4-0 silk sutures to route the entire flow into cerebral arteries. A temporary tie was placed on the carotid bifurcation and the proximal CCA was permanently ligated using 4-0 sutures. Before making a small arteriotomy, a suture connecting a weight (25 g) was secured around the cCCA. Then a microcatheter (PE-8-100, SAI Infusion Technologies) was flushed with 2% heparin (1,000 units/ml, heparin sodium, Upjohn), inserted into the ipsilateral CCA via the arteriotomy and advanced into the internal carotid artery. The catheter was secured by two purse-string suture ties around CCA.

### Interventional MRI

The mice with IA catheter secured in place were positioned in a Bruker 11.7T MRI scanner. Baseline T2 (TR/TE=2,500/30 ms), T1 (TR/TE 350/6.7 ms)-weighted and dynamic gradient echo echo-planar imaging (GE-EPI, TR/TE 1250/9.7 ms, field of view (FOV)=14×14 mm, matrix=128×128, acquisition time=60 s and 24 repetitions) images of the brain were acquired. The microcatheter was connected to a syringe mounted on an MRI compatible programmable syringe pump (PHD 2000, Harvard Apparatus Inc.) for controlled solution administration. Gadolinium (Gd; Prohance) dissolved in saline at 1:50 was infused intra-arterially at the rate of 0.15 ml/min under dynamic GE-EPI MRI for visualization of perfusion territory. For animals where the cortex was not perfused (most cases), the weight around cCCA was engaged, occluding the vessel with dynamic imaging of IA infusion to confirm cortical perfusion/supply.

Once cortical perfusion has been confirmed, 25% mannitol mixed with Gd (50:1) was infused until enhancement indicating BBB breach has been achieved (up to three bolus injections 1-2 min each; interval between infusions is 30 s). For detailed assessment of the BBB status, high resolution T1-weighted scan was collected after mannitol infusion. Three and seven days after OBBBO, the safety of the procedure was evaluated by MRI and then animals were sacrificed for further histological assessment.

### Cranial Window Implantation

Cranial window procedures were performed as previously described [22]. Briefly, mice were shaved and deeply anesthetized with 1.5-2% isoflurane, and stabilized on a stereotactic frame. Before surgery, animals were administered with dexamethasone sodium phosphate (0.02 ml at 4 mg/ml, Fresenius Kabi) by subcutaneous injection to prevent cerebral edema. Then the skin and periosteum were removed to expose the skull. A craniotomy (˜3 mm diameter) was conducted over the right parietal bone ˜1.5 mm posterior to bregma and ˜1.5 mm lateral from midline. Saline was applied regularly to avoid heating caused by drilling during skull-thinning procedure. At the end, the central island of skull bone was gently lifted, removed, and covered with a circular coverglass (3 mm diameter, #1 thickness, Harvard Biosciences) sealed to the skull using glue. For the subsequent imaging sessions, a custom-made head-bar with a circular opening was sealed to the skull with dental cement, covering all the exposed skull, wound margins and glass edges. Mice were allowed to recover for 7 days before imaging.

### Conjugation of BV and Fluorescein

Before labelling BV was washed 3 times using ultrafiltration with Millipore Amicon Ultra Centrifugal Filters 50 K (Milipore). After washing, the antibody was resuspended in saline at the concentration of 10 mg/ml and pH was adjusted to 9.0 with 0.1M Na₂CO₃. Then, NHS-Fluorescein (Thermo Fisher Scientific) dissolved in DMSO at the concertation of 10 mg/ml was mixed with antibody in the 1:10 molar ratio. Conjugation was carried for 30 min at RT and another 1 h in 37 C with 160 RPM agitation. The BV-FITC complexes were washed 3 times with saline on the 50 kDa centrifugal filters. Final protein concentration of BV-FITC was determined by absorbance at 280 nm measured with NanoDrop (Thermo Fisher Scientific).

### Matrix-Assisted Laser Desorption Ionization-Time-of-Flight (MALDI-TOF)

To determine the average number of fluorescein molecules conjugated with BV MALDI-TOF spectra of unmodified antibody and BV-FITC conjugate was recorded on a Voyager DE-STR spectrophotometer using 2,5-dihydroxybenzoic acid (DHB) as a matrix. First, protein samples were desalted using Zeba™ spin columns 7K MWCO (Thermo Fisher Scientific) and 10 μL elutions were mixed with 10 μL of matrix (10 mg/mL). Then 1 μL of this mixture was placed on the target plate in triplicate to dry. The mixture was redissolved in 50% methanol (MeOH) and 0.1% trifluoroacetate (TFA) aqueous solution. Number of shots and laser power was adjusted according to spectrum quality.

### Intravital Epi-Fluorescence and 2PM

Before microscopy, isoflurane anesthetized mice (n=5) with cranial window and with arterial access as described above were stabilized in a custom-made frame immobilizing their head. The mice were positioned under an epi-fluorescent microscope and a 10× magnification objective was used for capturing images at frequency of 1-2 Hz. Saline solution of 0.001 mM rhodamine (0.58 kDa) was injected using a syringe infusion pump at the rate of 0.15 ml/min over 1 min via the ICA microcatheter to visualize trans-catheter perfusion. When the cortex was not perfused, the cCCA was closed temporarily for 20 s by engaging the weights.

For 2PM to visualize OBBBO and drug penetration, mice were placed under a multiphoton microscope (FV1000MPE, Olympus, Tokyo, Japan). A 10× objective (UPlanSApo, 0.40 NA and 3.1 mm working distance) was centered over the cranial window and used to collect time series images of 800×800 pixels (1.59 μm/pixel; 2 μs/pixel; 100 frames) at an estimated depth of 150 um below the cortical surface. Rhodamine (0.002 mM) mixed with BV-FITC (0.01 mM) was injected prior to OBBBO to collect baseline data and optimize cortical perfusion (temporary cCCA closure). Then, 25% mannitol mixed with rhodamine and BV-FITC was delivered at the rate of 0.15 ml/min for 4 mins in total. A common excitation wavelength of 800 nm was used to simultaneously image both dyes during injection and dynamic imaging was continuously performed for 14 mins.

### Histology and Immunohistochemistry

For histological evaluation on the safety of OBBBO in the cortex, 7 days after surgery, animals (n=4) were anesthetized and perfused transcardially with 5% sucrose, followed by 4% paraformaldehyde (PFA). The brains were rapidly removed and post-fixed overnight in 4% PFA at 4° C. The brains were cryopreserved in 30% sucrose and 30-μm thick coronal sections were cryosectioned. Immunohistochemistry for anti-GFAP (1:250, Dako) and anti-Iba1 (1:250, Wako) was performed to assess the neuroinflammation. The secondary antibody was goat anti-rabbit (Alexa Fluor-488, 1:200, Molecular Probes). For detecting the biodistribution of infused BV, the mice with OBBBO (n=4) and without OBBBO (n=3) were sacrificed 1 hour after administration. The brains were cryosectioned at 30 μm and the slices were stained with goat anti-human secondary antibody (Alexa Fluor-488, 1:200, Invitrogen). All the fluorescent images were acquired using an inverted microscope (Zeiss, Axio Observer Z1).

### Image Processing and Statistical Analysis

Data is expressed as mean±SD unless otherwise specified. Quantitation of immunohistochemistry was based on relative fluorescence using Image J and analyzed using a paired t-test. The ratio of ipsi-/contralateral was analyzed using an unpaired t-test. The MRI analysis of the change in area of the Gd perfusion territory and Gd-enhancement for each mouse was calculated using a custom-written script in MATLAB and analyzed using a paired t-test. A p-value less than 0.05 was considered significant.

### Results

### Real-Time MRI Shows Variability of Cortical Trans-Catheter Perfusion

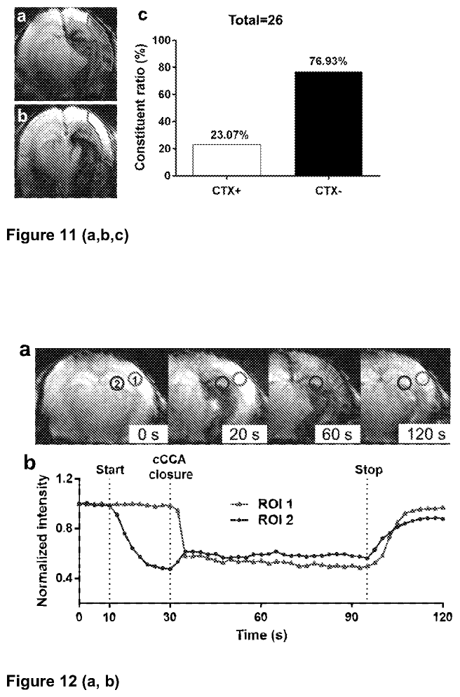

For the studied 26 mice we found that using infusion rate of 0.15 ml/min, which has been proven as a maximum safe speed, we observed variability in cortical involvement. Infusion of a contrast agent (Gd or SPIO) visualized the perfusion territory of the brain as hypointense regions on T2* MRI, which was sampled by GE-EPI scans at a temporal resolution of 2 volumes per second. Such real-time MRI allows precise spatiotemporal visualization of the parenchymal perfusion territory. IA infusion of Gd via ICA with dynamic GE-EPI imaging revealed T2* hypointensity in cerebral cortex (FIG. 11a) at a frequency of 23.07%. The lack of cortical perfusion using this delivery route (FIG. 11b) was observed much more frequently (76.93%). This phenomenon hints at variability of clinical outcomes and is an obvious obstacle complicating 2PM studies.

Temporary Closure of cCCA Facilitates Cortical Perfusion Visualized Under Real-Time MRI

Dynamic GE-EPI scans clearly visualized the biodistribution of IA injected contrast. In animals lacking trans-catheter perfusion through the cortex (drop of T2* signal) temporary closure of the cCCA redistributed the cerebral blood flow opening up the cortex for the catheter infusion (FIG. 12a). The dynamic signal changes for two selected ROIs are shown in FIG. 12b. There was a steep and early drop in the signal intensity (SI) in the hippocampus (ROI2), at that time SI in the cortex (ROI1) remained unchanged and dropped only after temporary closure of the cCCA.

### Osmotic Disruption of the BBB in Cerebral Cortex Using Real-Time MRI Guidance

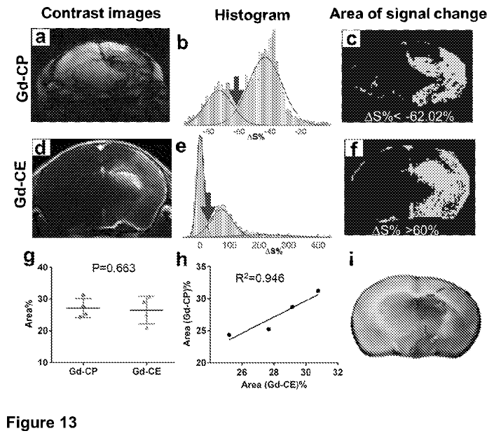

Immediately after confirming trans-catheter Gd-contrast perfusion (Gd—CP) in the cortex with IA infusion of the contrast agent (FIG. 13a), IA mannitol was infused using the same parameters. Effective BBBO was reflected by Gd-contrast enhancement (Gd-CE) on the T1-weighted scan in the region previously highlighted by the contrast infusion (FIG. 13d). To determine the correlation between the Gd—CP (FIG. 13a) and Gd-CE (FIG. 13b) MRI, the histograms were drawn and fitted into two Gaussian distributions (FIG. 13b,e). The values that corresponded to the minimal overlap between the two Gaussian functions were chosen to be the threshold that separated the pixels with a significant signal change. Using these thresholds, the areas with significant signal change were determined (FIG. 13c,f). For the four mice studied, the Gd—CP MRI showed an average signal change area of 27.13±2.36%, while Gd-CE showed an average signal change area of 26.50±3.40%, which was not significantly different (P=0.663, FIG. 13g). A good correlation was shown between these two methods (R²=0.946, FIG. 13h). This indicated a successful OBBBO in cortex by IA mannitol, as predicted by the perfusion pre-scan. Furthermore, the histopathological validation using Evans blue, which is state of the art technique for BBB assessment, displayed a pattern of extravasation that was consistent with MRI (FIG. 13i).

### Safety and Long-Term Consequences of IA Mannitol-Induced BBBO in the Cortex

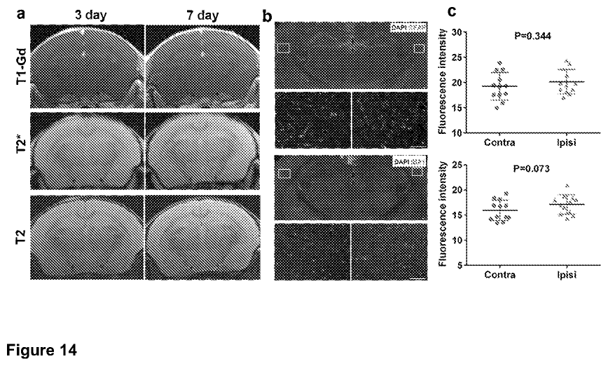

Three and seven days after BBBO, T2w MRI did not detect any asymmetry or hyperintensity, suggesting a lack of edema or inflammation, T2*w scans were not indicative of microhemorrhages and a lack of Gd-enhancement on T1w images revealed an intact BBB (FIG. 14a), overall suggesting that the procedure is safe and the BBB breach was transient. Histology corroborated these observations with GFAP and IBA-1 staining 7 days post BBBO, in which there was no evidence of astrocytic or microglial activation in the BBBO region, as determined by comparing the fluorescence intensity between the targeted region and the corresponding area in the contralateral hemisphere (P=0.344, P=0.073; FIG. 14b,c). Overall, both MRI and histologic appearance confirmed that the procedure for cortical BBBO induction did not cause brain damage. Notably, excessive exposure to IA mannitol i.e. continuous 4 min-infusion led to brain damage, the injury was shown as T2 hyperintensity.

### Vascular Trans-Catheter Perfusion in Mouse Cortex Through a Cranial Window

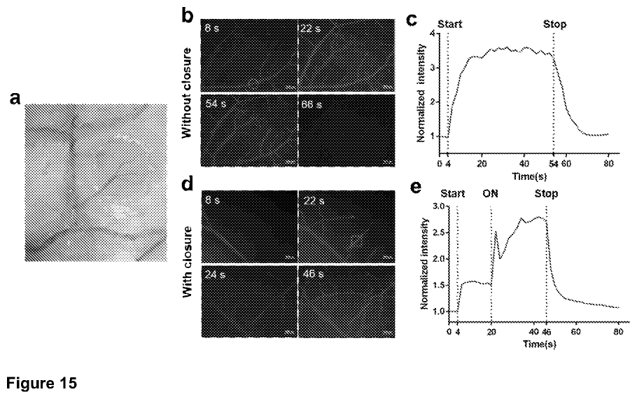

With the goal of developing a protocol enabling comprehensive assessment of cortical BBB, including intravital microscopy, we implanted cranial windows and head posts (n=5) (FIG. 15a). After allowing the animals to heal for one week, the mice were catheterized intra-arterially and placed under epi-fluorescent microscopy. Rhodamine was infused via the catheter to verify perfusion and display the cortical vascular architecture. In an agreement with the observation under MRI, cortical perfusion was observed rarely, as visualized during IA infusion bolus of rhodamine (FIG. 15b). In those animals, the dynamic signal changes showed steep increase for the duration of bolus infusion consistently for cortical vessels (FIG. 15c). In the majority of animals, however, sparse or none cerebral arteries and microvessels were perfused and temporary cCCA closure needed to be performed to rapidly increase and broaden perfusion territory in the cortex (FIG. 15d). The dynamic assessment of that scenario is quantitatively represented in FIG. 15e.

### Intravital Multiphoton Microscopy for Visualization of Cortical BBBO and Drug Extravasation

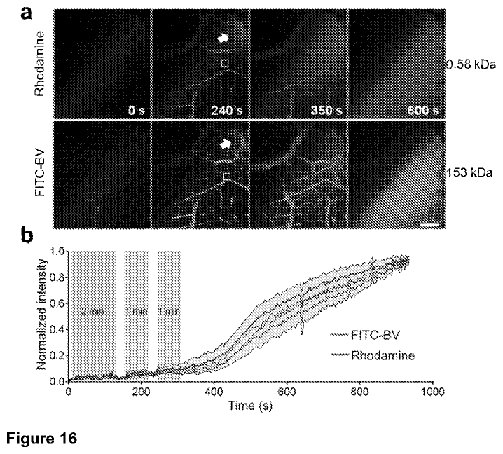

The cerebral vasculature at ˜100 μm depth into the cortex was visualized with 2PM upon IA injection of rhodamine. Once cortical perfusion was achieved, infusion (2 min IA bolus) of a mixture of mannitol, rhodamine and BV-FITC was initiated; however infiltration was not observed. Subsequently, another infusion (1 min bolus) was performed, the BBB was breached, and a final infusion (1 min bolus) was performed, for a total of 4 minutes of infusion time, which resulted in a more robust penetration into the cortical parenchyma (FIG. 16a). The 0.58 kDa rhodamine extravasated the cortex earlier compared to 153 kDa BV-FITC. The fluorescence intensity changes in 7 selected ROIs located in the parenchyma was measured to exhibit dynamics of BBB permeability for rhodamine and BV-FITC. As anticipated, there was earlier onset and higher intensity of extravasation for rhodamine upon BBBO compared to BV-FITC (FIG. 16b).

### Histological Confirmation of BV Extravasation

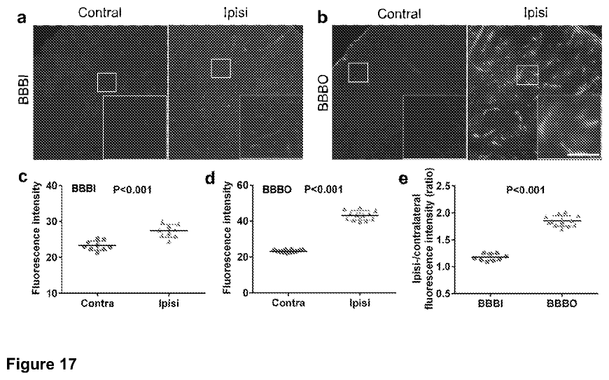

Cryosectioned brain tissue samples collected one hour after IA delivery of BV with intact BBB (BBBI) showed modestly increased uptake of BV delivery to the target (ipsilateral side) but it was localized within the blood vessels. (FIG. 17a). For the IA delivery with OBBBO, accumulation of BV was observed in both blood vessels and parenchyma. Additionally, OBBBO appeared to potentiate the vascular concentration of BV. As measured by the fluorescence intensity, there was significantly higher uptake of BV in ipsilateral vs. contralateral hemisphere in both groups (P<0.001, FIG. 17b), but the ipsi-/contralateral ratio was more pronounced when the BBB was opened (P<0.001, FIG. 17e). All the observations demonstrated that IA delivery of BV into the brain across an osmotically opened BBB is more effective compared to the intact BBB (BBBI).

### Discussion

Intra-arterial hyperosmotic mannitol has been used to induce transient permeabilization of the BBB for enhancing drug delivery to the brain. However, due to the unpredictable and non-selective opening, this approach was linked with high variability of outcomes [12, 16], preventing its broad clinical adaptation. Our previous studies have proved the superiority of real-time MRI guidance, facilitating highly predictable and spatially precise endovascular targeting of the brain to induce OBBBO and deliver therapeutics [13, 14, 19, 23]. There is growing demand for this type of technology due to the rapidly growing field of endovascular neurointerventions. Indeed, we have recently applied this approach clinically in a patient with aggressive recurrent glioblastoma multiforme. Real-time MRI guidance of IA delivery was essential to maximize tumor exposure with BV following mannitol infusion, resulting in encouraging therapeutic response [16]. Additionally, our PET imaging study demonstrated that IA route is far more effective in delivering monoclonal antibody into the brain compared to systemic administration and the antibody was retained in the brain for at least 24 hours [15]. However, that study only presented the relatively low spatial resolution PET data precluding assessment whether the accumulation was solely on the endothelial level or the antibody penetrated into the brain parenchyma. Here, we focused on optimizing IA drug delivery in mice to facilitate multi-scale dynamic imaging studies of BBBO, particularly for intravital microscopy of drug extravasation.

OBBBO in mice has been previously reported and several studies showed successful BBB breach in the entire hemisphere including the cortex [10, 24, 25]; however, these published studies utilized a high IA infusion rate exceeding the safe physiological perfusion rate for the carotid artery, and it has been reported by us and others that excessive infusion rate has a direct damaging effect on the BBB and the brain [19, 23, 26, 27]. We previously optimized the procedure for safe, transient opening of the BBB without neurological consequences but the territory of BBB opening rarely included the cortex. This phenomenon is likely due to redundancy in vascularization of the cortex supplied by more than one major cerebral artery eventually leading to mixing and dilution of IA mannitol [28, 29]. In order to prevent this situation, here, we temporarily occluded the cCCA for the duration of mannitol injection and that intervention was sufficient for the ipsilateral cortex to be perfused from the catheter and therefore disrupt the cortical BBB as shown by real-time MRI. This experimental platform was then exploited for studying the mechanism of drug extravasation using intravital microscopy. Dynamic imaging during IA infusion allowed us to visualize and track the leakage of fluorescent dyes upon BBBO, showing that rhodamine extravasated earlier and led to significantly higher parenchymal accumulation than monoclonal antibody. This observation is consistent with a study of focused ultrasound (FUS)-induced BBBO reported by Nhan. et al that fast leakage for small sized molecules [30]. Indeed, FUS is emerging as a novel non-invasive technology for BBB opening to enhance delivery of therapeutics into the brain [31-34]. This approach, especially when performed under MRI-guidance, has excellent spatial control; however, the strategy needs to overcome the sterile inflammatory response before being widely implemented in clinical trials [35]. Furthermore, FUS-induced BBBO in the brain parenchyma usually is combined with systemic administration of therapeutics, making it difficult to reach sufficient drug concentration at the targeted site and often resulting in toxic side effects. In contrast, IA approach combining selective OBBBO immediately followed by localized delivery of a specific drug during the same procedure as a one-stop-shop affords adequate therapeutic concentration at the desired destination while minimizing systemic exposure.

Microscopic analysis in this study (both intravital and post mortem) provided information about the timing of BBB breach as well as parenchymal penetration of injected antibodies, further explaining our previous PET findings [15] and other literature reports [7, 36, 37]. After IA delivery with intact BBB antibodies were found localized to the blood vessels, while parenchymal presence was negligible. This is consistent with published literature showing extravasation of antibodies without BBBO is marginal [15, 38-40]. Notably, OBBBO and intravenous delivery of antibodies also results in poor brain accumulation [15].

IA mannitol with coordinated closure of cCCA facilitated cortical BBBO; however, for effective BBB disruption longer exposure to mannitol (around 3 minutes) was required compared to subcortical structures. This phenomenon may result from the mixing and dilution of mannitol or differences in structure and function of cortical capillaries. In support of the mixing theory is our dynamic intravital microscopy where we observed the intermittent pulsatile flow pattern during IA infusion of the contrast agent. Structure and function of the microvessels may also contribute to differences in vulnerability to mannitol as it has been shown in the in vitro BBB model based on human iPSC-derived brain microvascular endothelial cells (dhBMECs), where the mannitol-induced BBB disruption was not homogenous [41].

The multi-scale imaging studies reported here are essential for developing precise, reproducible, and effective strategies for drug targeting. Even in case of direct intracerebral injection of small molecules, based on convection-enhanced delivery (CED), drug retention in the brain is uncertain. A recent PET study surprisingly reported that CED of low molecular weight molecules resulted in their rapid clearance [42]. The mechanism of that rapid clearance is not well understood but the BBB functionality includes active efflux transporting molecules out of the CNS [43]. Meanwhile, this finding might also explain the limited efficacy of therapies when BBB permeable small molecules were used to treat CNS disorders, as they seem to be easily transported out, resulting in inadequate therapeutic concentrations at the target. Hence, our developed platform for intravital imaging in the cortex will be of great value to accurately understand the drug behavior in the brain parenchyma with or without BBBO, profoundly contributing to the development of drug delivery strategies.

Overall, this study established reproducible cortical BBBO in mice, which enables multi-photon microscopy studies on BBBO and drug targeting. This approach enabled the real-time monitoring of the extravasation of IA injected antibodies.

### REFERENCES FOR EXAMPLE 3

[1] G. W. Goldstein, A. L. Betz, The Blood-Brain-Barrier, Sci Am, 255 (1986) 74-&.
[2] W. M. Pardridge, The blood-brain barrier: bottleneck in brain drug development, NeuroRx, 2 (2005) 3-14.
[3] R. K. Oberoi, K. E. Parrish, T. T. Sio, R. K. Mittapalli, W. F. Elmquist, J. N. Sarkaria, Strategies to improve delivery of anticancer drugs across the blood-brain barrier to treat glioblastoma, Neuro Oncol, 18 (2016) 27-36.
[4] V. K. Gribkoff, L. K. Kaczmarek, The need for new approaches in CNS drug discovery: Why drugs have failed, and what can be done to improve outcomes, Neuropharmacology, 120 (2017) 11-19.
[5] W. A. Banks, From blood-brain barrier to blood-brain interface: new opportunities for CNS drug delivery, Nat Rev Drug Discov, 15 (2016) 275-292.
[6] K. Aldape, K. M. Brindle, L. Chesler, R. Chopra, A. Gajjar, M. R. Gilbert, N. Gottardo, D. H. Gutmann, D. Hargrave, E. C. Holland, D. T. W. Jones, J. A. Joyce, P. Kearns, M. W. Kieran, I. K. Mellinghoff, M. Merchant, S. M. Pfister, S. M. Pollard, V. Ramaswamy, J. N. Rich, G. W. Robinson, D. H. Rowitch, J. H. Sampson, M. D. Taylor, P. Workman, R. J. Gilbertson, Challenges to curing primary brain tumours, Nat Rev Clin Oncol, (2019).
[7] S. I. Rapoport, Advances in osmotic opening of the blood-brain barrier to enhance CNS chemotherapy, Expert Opin Investig Drugs, 10 (2001) 1809-1818.
[8] D. F. Kraemer, D. Fortin, E. A. Neuwelt, Chemotherapeutic dose intensification for treatment of malignant brain tumors: recent developments and future directions, Curr Neurol Neurosci Rep, 2 (2002) 216-224.
[9] W. G. Lesniak, C. Chu, A. Jablonska, B. Behnam Azad, O. Zwaenepoel, M. Zawadzki, A. Lisok, M. G. Pomper, P. Walczak, J. Gettemans, M. Janowski, PET imaging of distinct brain uptake of a nanobody and similarly-sized PAMAM dendrimers after intra-arterial administration, Eur J Nucl Med Mol Imaging, (2019).
[10] C. P. Foley, D. G. Rubin, A. Santillan, D. Sondhi, J. P. Dyke, R. G. Crystal, Y. P. Gobin, D. J. Ballon, Intra-arterial delivery of AAV vectors to the mouse brain after mannitol mediated blood brain barrier disruption, J Control Release, 196 (2014) 71-78.
[11] S. Cerri, R. Greco, G. Levandis, C. Ghezzi, A. S. Mangione, M. T. Fuzzati-Armentero, A. Bonizzi, M. A. Avanzini, R. Maccario, F. Blandini, Intracarotid Infusion of Mesenchymal Stem Cells in an Animal Model of Parkinson's Disease, Focusing on Cell Distribution and Neuroprotective and Behavioral Effects, Stem Cells Transl Med, 4 (2015) 1073-1085.
[12] S. Joshi, A. Ergin, M. Wang, R. Reif, J. Zhang, J. N. Bruce, I. J. Bigio, Inconsistent blood brain barrier disruption by intraarterial mannitol in rabbits: implications for chemotherapy, J Neurooncol, 104 (2011) 11-19.
[13] P. Walczak, J. Wojtkiewicz, A. Nowakowski, A. Habich, P. Holak, J. Xu, Z. Adamiak, M. Chehade, M. S. Pearl, P. Gailloud, B. Lukomska, W. Maksymowicz, J. W. Bulte, M. Janowski, Real-time MRI for precise and predictable intra-arterial stem cell delivery to the central nervous system, J Cereb Blood Flow Metab, (2016).
[14] M. Janowski, P. Walczak, M. S. Pearl, Predicting and optimizing the territory of blood-brain barrier opening by superselective intra-arterial cerebral infusion under dynamic susceptibility contrast MRI guidance, Journal of Cerebral Blood Flow & Metabolism, 36 (2016) 569-575.
[15] W. G. Lesniak, C. Chu, A. Jablonska, Y. Du, M. G. Pomper, P. Walczak, M. Janowski, PET imaging of intra-arterial (89)Zr bevacizumab in mice with and without osmotic opening of the blood-brain barrier: distinct advantage of intra-arterial delivery, J Nucl Med, (2018).
[16] M. Zawadzki, J. Walecki, B. Kostkiewicz, K. Kostyra, M. S. Pearl, M. Solaiyappan, P. Walczak, M. Janowski, Real-time MRI guidance for intra-arterial drug delivery in a patient with a brain tumor: technical note, BMJ Case Rep, 12 (2019).
[17] K. Svoboda, R. Yasuda, Principles of two-photon excitation microscopy and its applications to neuroscience, Neuron, 50 (2006) 823-839.
[18] Y. Liang, K. Li, K. Riecken, A. Maslyukov, D. Gomez-Nicola, Y. Kovalchuk, B. Fehse, O. Garaschuk, Long-term in vivo single-cell tracking reveals the switch of migration patterns in adult-born juxtaglomerular cells of the mouse olfactory bulb, Cell Res, 26 (2016) 805-821.
[19] C. Chu, G. Liu, M. Janowski, J. W. M. Bulte, S. Li, M. Pearl, P. Walczak, Real-Time MRI Guidance for Reproducible Hyperosmolar Opening of the Blood-Brain Barrier in Mice, Front Neurol, 9 (2018) 921.
[20] G. Makowicz, R. Poniatowska, M. Lusawa, Variants of cerebral arteries—anterior circulation, Pol J Radiol, 78 (2013) 42-47.
[21] D. S. Liebeskind, Collateral circulation, Stroke, 34 (2003) 2279-2284.
[22] A. Holtmaat, T. Bonhoeffer, D. K. Chow, J. Chuckowree, V. De Paola, S. B. Hofer, M. Hubener, T. Keck, G. Knott, W. C. Lee, R. Mostany, T. D. Mrsic-Flogel, E. Nedivi, C. Portera-Cailliau, K. Svoboda, J. T. Trachtenberg, L. Wilbrecht, Long-term, high-resolution imaging in the mouse neocortex through a chronic cranial window, Nat Protoc, 4 (2009) 1128-1144.
[23] R. Guzman, M. Janowski, P. Walczak, Intra-Arterial Delivery of Cell Therapies for Stroke, Stroke, 49 (2018) 1075-1082.
[24] M. Kaya, S. Gulturk, I. Elmas, R. Kalayci, N. Arican, Z. C. Kocyildiz, M. Kucuk, H. Yorulmaz, A. Sivas, The effects of magnesium sulfate on blood-brain barrier disruption caused by intracarotid injection of hyperosmolar mannitol in rats, Life Sci, 76 (2004) 201-212.
[25] E. K. Weidman, C. P. Foley, O. Kallas, J. P. Dyke, A. Gupta, A. E. Giambrone, J. Ivanidze, H. Baradaran, D. J. Ballon, P. C. Sanelli, Evaluating Permeability Surface-Area Product as a Measure of Blood-Brain Barrier Permeability in a Murine Model, AJNR Am J Neuroradiol, 37 (2016) 1267-1274.
[26] M. Janowski, A. Lyczek, C. Engels, J. Xu, B. Lukomska, J. W. Bulte, P. Walczak, Cell size and velocity of injection are major determinants of the safety of intracarotid stem cell transplantation, J Cereb Blood Flow Metab, 33 (2013) 921-927.
[27] L. L. Cui, E. Kerkela, A. Bakreen, F. Nitzsche, A. Andrzejewska, A. Nowakowski, M. Janowski, P. Walczak, J. Boltze, B. Lukomska, J. Jolkkonen, The cerebral embolism evoked by intra-arterial delivery of allogeneic bone marrow mesenchymal stem cells in rats is related to cell dose and infusion velocity, Stem Cell Res Ther, 6 (2015) 11.
[28] L. A. Gillilan, Potential collateral circulation to the human cerebral cortex, Neurology, 24 (1974) 941-948.
[29] E. Cuccione, G. Padovano, A. Versace, C. Ferrarese, S. Beretta, Cerebral collateral circulation in experimental ischemic stroke, Exp Transl Stroke Med, 8 (2016) 2.
[30] T. Nhan, A. Burgess, E. E. Cho, B. Stefanovic, L. Lilge, K. Hynynen, Drug delivery to the brain by focused ultrasound induced blood-brain barrier disruption: quantitative evaluation of enhanced permeability of cerebral vasculature using two-photon microscopy, J Control Release, 172 (2013) 274-280.
[31] A. Burgess, S. Dubey, S. Yeung, O. Hough, N. Eterman, I. Aubert, K. Hynynen, Alzheimer disease in a mouse model: MR imaging-guided focused ultrasound targeted to the hippocampus opens the blood-brain barrier and improves pathologic abnormalities and behavior, Radiology, 273 (2014) 736-745.
[32] K. T. Chen, K. C. Wei, H. L. Liu, Theranostic Strategy of Focused Ultrasound Induced Blood-Brain Barrier Opening for CNS Disease Treatment, Front Pharmacol, 10 (2019) 86.
[33] A. B. Etame, R. J. Diaz, C. A. Smith, T. G. Mainprize, K. Hynynen, J. T. Rutka, Focused ultrasound disruption of the blood-brain barrier: a new frontier for therapeutic delivery in molecular neurooncology, Neurosurg Focus, 32 (2012) E3.
[34] S. Wang, I. S. Shin, H. Hancock, B. S. Jang, H. S. Kim, S. M. Lee, V. Zderic, V. Frenkel, I. Pastan, C. H. Paik, M. R. Dreher, Pulsed high intensity focused ultrasound increases penetration and therapeutic efficacy of monoclonal antibodies in murine xenograft tumors, J Control Release, 162 (2012) 218-224.
[35] Z. I. Kovacs, S. Kim, N. Jikaria, F. Qureshi, B. Milo, B. K. Lewis, M. Bresler, S. R. Burks, J. A. Frank, Disrupting the blood-brain barrier by focused ultrasound induces sterile inflammation, Proc Natl Acad Sci USA, 114 (2017) E75-E84.
[36] E. A. Neuwelt, J. Minna, E. Frenkel, P. A. Barnett, C. I. McCormick, Osmotic blood-brain barrier opening to IgM monoclonal antibody in the rat, Am J Physiol, 250 (1986) R875-883.
[37] B. Wang, T. Siahaan, R. Soltero, Drug Delivery: Principles and Applications, Wiley Ser Drug Disc, (2005) 1-448.
[38] R. Razpotnik, N. Novak, V. Curin Serbec, U. Rajcevic, Targeting Malignant Brain Tumors with Antibodies, Front Immunol, 8 (2017) 1181.
[39] R. T. Frank, K. S. Aboody, J. Najbauer, Strategies for enhancing antibody delivery to the brain, Biochim Biophys Acta, 1816 (2011) 191-198.
[40] H. L. Liu, P. H. Hsu, C. Y. Lin, C. W. Huang, W. Y. Chai, P. C. Chu, C. Y. Huang, P. Y. Chen, L. Y. Yang, J. S. Kuo, K. C. Wei, Focused Ultrasound Enhances Central Nervous System Delivery of Bevacizumab for Malignant Glioma Treatment, Radiology, 281 (2016) 99-108.
[41] R. M. Linville, J. G. DeStefano, M. B. Sklar, Z. Xu, A. M. Farrell, M. I. Bogorad, C. Chu, P. Walczak, L. Cheng, V. Mahairaki, K. A. Whartenby, P. A. Calabresi, P. C. Searson, Human iPSC-derived blood-brain barrier microvessels: validation of barrier function and endothelial cell behavior, Biomaterials, 190-191 (2019) 24-37.
[42] U. Tosi, H. Kommidi, V. Bellat, C. S. Marnell, H. Guo, O. Adeuyan, M. E. Schweitzer, N. Chen, T. Su, G. Zhang, U. B. Maachani, D. J. Pisapia, B. Law, M. M. Souweidane, R. Ting, Real-Time, in Vivo Correlation of Molecular Structure with Drug Distribution in the Brain Striatum Following Convection Enhanced Delivery, ACS Chem Neurosci, 10 (2019) 2287-2298.
[43] W. G. Mayhan, D. D. Heistad, Permeability of blood-brain barrier to various sized molecules, Am J Physiol, 248 (1985) H712-718.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the invention.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

It is understood that the detailed examples and embodiments described herein are given by way of example for illustrative purposes only, and are in no way considered to be limiting to the invention. Various modifications or changes in light thereof will be suggested to persons skilled in the art and are included within the spirit and purview of this application and are considered within the scope of the appended claims. For example, the relative quantities of the ingredients may be varied to optimize the desired effects, additional ingredients may be added, and/or similar ingredients may be substituted for one or more of the ingredients described. Additional advantageous features and functionalities associated with the systems, methods, and processes of the present invention will be apparent from the appended claims. Moreover, those skilled in the art will recognize, or be able to ascertain using no more than routine experimentation, many equivalents to the specific embodiments of the invention described herein. Such equivalents are intended to be encompassed by the following claims.

1. A method for treating a subject, comprising
(a) administering to brain tissue of a subject an effective amount of one or more therapeutic agents;
(b) imaging the subject to assess the one or more administered therapeutic agents.

2. The method of claim 1 wherein one or more therapeutic agents are administered through the subject's blood-brain barrier.

3. The method of claim 1 wherein the subject is imaged during or after the administration of the one or more therapeutic agents.

4. The method of claim 1 wherein the subject's brain tissue is imaged.

5. The method of claim 1 wherein the one or more therapeutic agents are administered by systemically, intraarterially or parenchymal injection.

6. The method of claim 1 wherein uptake or clearance of the administered therapeutic agents are assessed.

7. The method of claim 6 wherein administration of the therapeutic agents is modified based on of the assessment.

8. The method of claim 7 wherein administration rates, administration duration or dosages of the one or more therapeutic agents are modified based on the assessment.

9. The method of claim 1 wherein the imaging comprises positron-emission tomography (PET).

10. The method of claim 9 wherein the imaging comprises dynamic PET scans and/or whole body dynamic PET/CT imaging and or dynamic PET/MRI.

11-17. (canceled)

18. The method of claim 1 wherein the subject's blood-brain barrier is disrupted prior to administering the one or more therapeutic agents.

19-22. (canceled)

23. The method of claim 1 wherein the subject's blood-brain barrier is not disrupted prior to administering the one or more therapeutic agents.

24-25. (canceled)

26. The method of claim 1 wherein magnetic resonance images are acquired while the one or more therapeutic agents are administered.

27. A method for treating a subject, comprising:
(a) administering to a subject a combination of an effective amount of 1) one or more blood-brain barrier (BBB) opening agents and 2) one or more contrast agents to thereby disrupt the blood-brain barrier of the subject;
(b) imaging the subject's blood-brain barrier;
(c) administering to a subject an effective amount of one or more therapeutic agents through the subject's blood-brain barrier;
(d) imaging the subject to assess the one or more administered therapeutic agents.

28-30. (canceled)

31. The method of claim 1 wherein infusion parameters are adjusted based on imaging following the administering of the one or more therapeutic agents.

32. The method of claim 1 wherein dose and/or distribution of the one or more therapeutic agents are adjusted based on imaging data obtained following the administering of the one or more therapeutic agents.

33. The method of claim 1 wherein the subject is a human.
